(19) United States
(12) Patent Application Publication
(10) Pub. No.: US 2003/0036643 A1
(43) Pub. Date: Feb. 20, 2003

(54) Methods and compositions for the construction and use of fusion libraries

(76) Inventors: Cheng He Jin, San Diego, CA (US); Min Li, Lutherville, MD (US); Hong-Xiang Liu, Monrovia, CA (US); Christian Melander, Monrovia, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/953,351

(22) Filed: Sep. 14, 2001

Related U.S. Application Data
(60) Provisional application No. 60/232,960, filed on Sep. 14, 2000.

(51) Int. Cl. C07H 21/02; C07H 21/04

(52) U.S. Cl. 536/23.1

(57) ABSTRACT

This invention pertains to genetic libraries encoding enzyme fusion proteins and methods of use to identify a nucleic acid of interest.

This is a continuing application of 60/232,960 filed on Sep. 14, 2000.

### FIELD OF THE INVENTION

This invention pertains to genetic libraries encoding enzyme fusion proteins and methods of use to identify a nucleic acid of interest.

### BACKGROUND OF THE INVENTION

Improvements in DNA technology and bioinformatics have enabled the raw genomic sequences of a few microorganisms to be made available to the scientific community, and the sequencing of genomes of higher eukaryotes and mammals are nearly completed. The rapid accumulation of DNA sequences from various organisms presents tremendous potential scientific and commercial opportunities. However, in many cases, the available raw sequences cannot be translated into knowledge of their encoded biological, pharmaceutical or industrial usefulness. Thus, there is a need in the art for technologies that will efficiently, systematically, and maximally realize the function and utility of DNA sequences from both natural and synthetic sources.

Several general approaches to realize the potential functions of a given DNA sequence have been reported. One approach, which is also the primary approach in gene and target discovery, is to rely on bioinformatic tools. Bioinformatics software is available from a number of companies specializing in organization of sequence data into computer databases. A researcher is able to compare uncharacterized nucleic acid sequences with the sequences of known genes in the database, thereby allowing theories to be proposed regarding the function of the nucleic acid sequence of an encoded gene product. However, bioinformatics software can be expensive, often requires extensive training for meaningful use, and enables a researcher to only speculate as to a possible function of an encoded gene product. Moreover, an increasing number of DNA sequences have been identified that show no sequence relationship to genes of known functions and new properties have been discovered for many so-called “known” genes. Therefore, bioinformatics provides a limited amount of information that must be used with caution. All informatics-predicted properties require experimental approval.

Another approach for associating function with sequence data is to pursue experimental testing of orphan gene function. In previously described methods, nucleic acid sequences are expressed using any of a number of expression constructs to obtain an encoded peptide, which is then subjected to assays to identify a peptide having a desired property. An inherent difficulty with many of the previously described methods is correlating a target property with its coding nucleic acid sequence. In other words, as large collections of nucleic acid and peptide sequences are gathered and their encoded functions explored, it is increasingly difficult to identify and isolate a coding sequence responsible for a desired function.

The fundamental difficulties associated with working with large collections of nucleic acid sequences, such as genetic libraries, are alleviated by linking the expressed peptide with the genetic material which encodes it. An approach of associating a peptide to its coding nucleic acid is the use of polysome display. Polysome display methods essentially comprise translating RNA in vitro and complexing the nascent protein to its corresponding RNA. The complex is constructed by manipulating the coding sequence such that the ribosome does not release the nascent protein or the RNA. By retrieving proteins of interest, the researcher retrieves the corresponding RNA, and thereby obtains the coding DNA sequence after converting the RNA into DNA via known methods such as reverse transcriptase-coupled PCR. Yet, polysome display methods can be carried out only in vitro, are difficult to perform, and require an RNase-free environment. Due to alternative starting methionine codons and the less than perfect processive nature of in vitro translation machinery, this method is not applicable to large proteins. In addition, the RNA-protein-ribosome complex is unstable, thereby limiting screening methods and tools suitable for use with polysome display complexes.

Another commonly used method of linking proteins to coding nucleic acid molecules for use with genetic libraries involves displaying proteins on the outer surface of cells, viruses, phages, and yeast. By expressing the variant protein as, for example, a component of a viral coat protein, the protein is naturally linked to its coding DNA located within the viral particle or cellular host, which can be easily isolated. The DNA is then purified and analyzed. Other systems for associating a protein with a DNA molecule in genetic library construction have been described in, for example, International Patent Applications WO 93/08278, WO 98/37186, and WO 99/11785. Yet, these approaches have features that are not most desirable. First, the expressed protein and the corresponding cDNA are non-covalently bound. The resulting complex is not stable or suitable for many selection procedures. Second, the display systems by design are restricted to either in vitro or prokaryotic heterologous expression systems, which may not provide necessary protein modification or folding machinery for the study of eukaryotic peptides. Incorrectly folded or modified proteins often lack the native function of desired proteins and are often very unstable. Third, if displayed on the surface of a biological particle, the expressed proteins often undergo unwanted biological selections intrinsic to the displayed systems. For example, in the case of display proteins on bacterial viruses, e.g., bacteriophage, the expressed protein will be assembled as part of bacterial virus coat proteins and displayed on the surface of the bacterial virus. Interactions of the bacterial virus-bound variant protein with the surrounding environment and incorporation of the protein into the bacterial viral coat can damage the conformation and activity of the variant protein. Moreover, even if the protein is incorporated into the bacterial viral capsid, the display protein may not be in a correct geometrical or stoichiometrical form, which is required for its activity. Fourth, construction of large surface-display libraries using biological particles is time intensive, and the researcher must take precautions to ensure that the biological particle, i.e., virus or phage, remains viable. Fifth, it is known that different hosts have different codon preferences when performing protein translation. For example, in prokaryotic systems, the expression systems used for bacterial virus display, there are at least five codons commonly recognized in mammalian cells that are not readily recognized by bacteria during protein translation. Thus, mammalian sequences with these codons are not translated or are translated very inefficiently in bacteria, posing a significant negative selection.

In view of the above, there remains a need in the art for a genetic library which allows easy association of a variant or unknown peptide and its coding sequence and methods of use. The invention provides such a library and method. In addition, the present invention allows the identification of relevant proteins in the native cellular environment, which is a significant advantage of the use of eucaryotic systems. These and other advantages of the present invention, as well as additional inventive features, will be apparent from the description of the invention provided herein.

### SUMMARY OF THE INVENTION

In accordance with the objects outlined herein *WILL FILL IN WHEN CLAIMS ARE FINALIZED

### DETAILED DESCRIPTION OF THE DRAWINGS

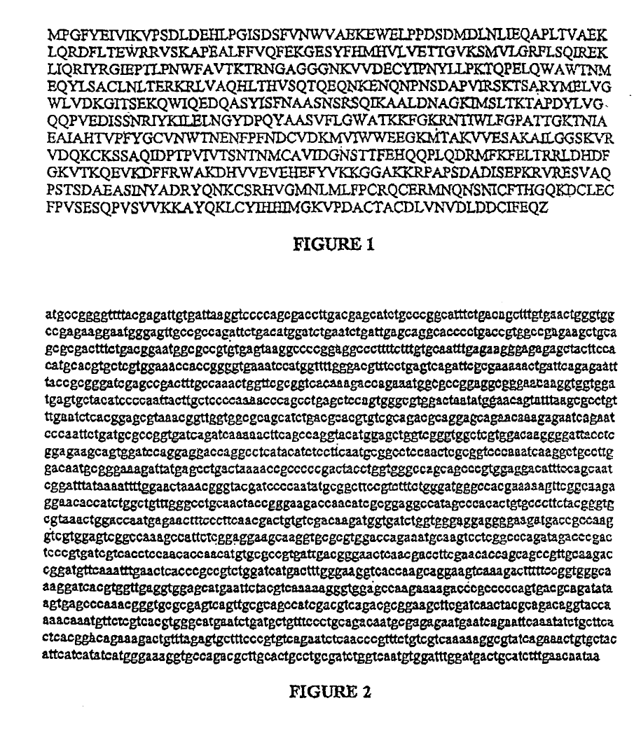

FIG. 1 depicts the amino acid sequence of Rep78 isolated from adeno-associated virus 2.

FIG. 2 depicts the nucleotide sequence of Rep78 isolated from adeno-associated virus 2.

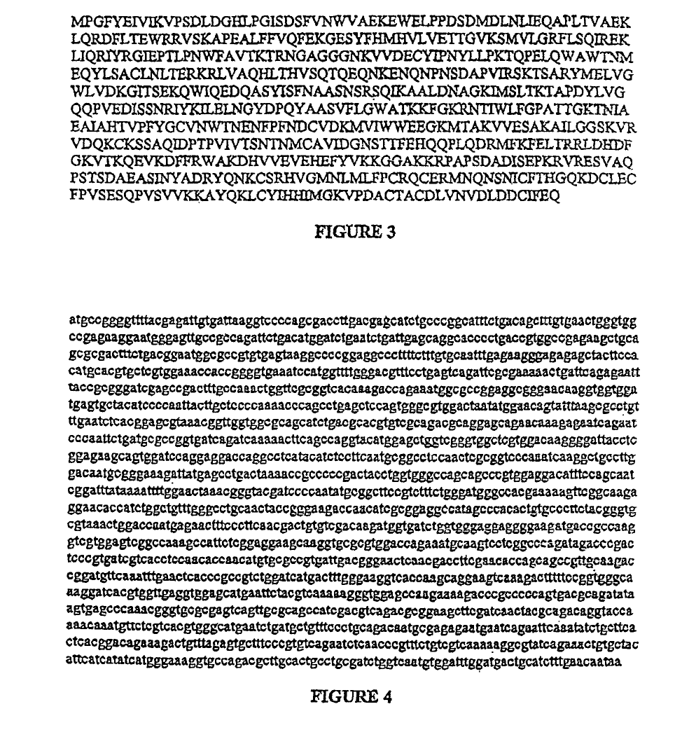

FIG. 3 depicts the amino acid sequence of major coat protein A isolated from adeno-associated virus 2.

FIG. 4 depicts the nucleotide sequence of major coat protein A isolated from adeno-associated virus 2.

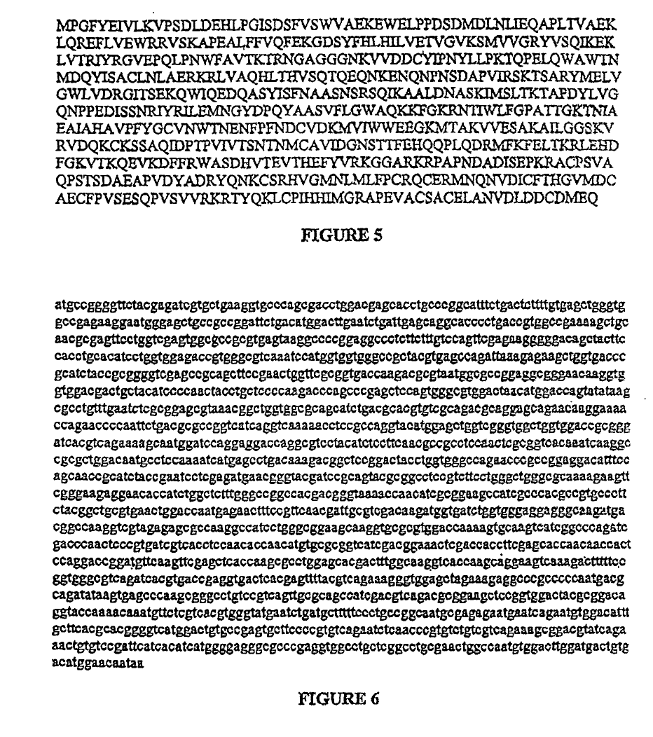

FIG. 5 depicts the amino acid sequence of a Rep protein isolated from adeno-associated virus 4.

FIG. 6 depicts the nucleotide sequence of a Rep protein isolated from adeno-associated virus 4.

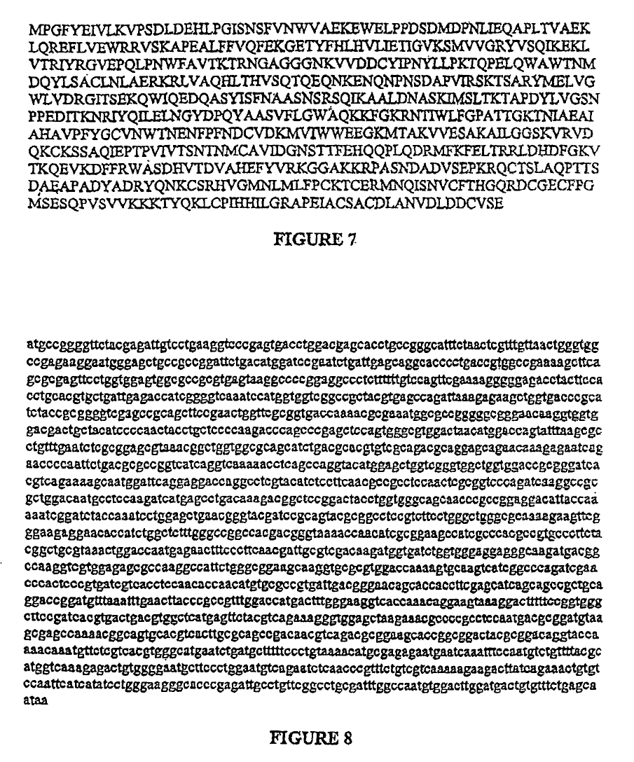

FIG. 7 depicts the amino acid sequence of Rep78 isolated from adeno-associated virus 3B.

FIG. 8 depicts the nucleotide sequence of Rep78 isolated from adeno-associated virus 3B.

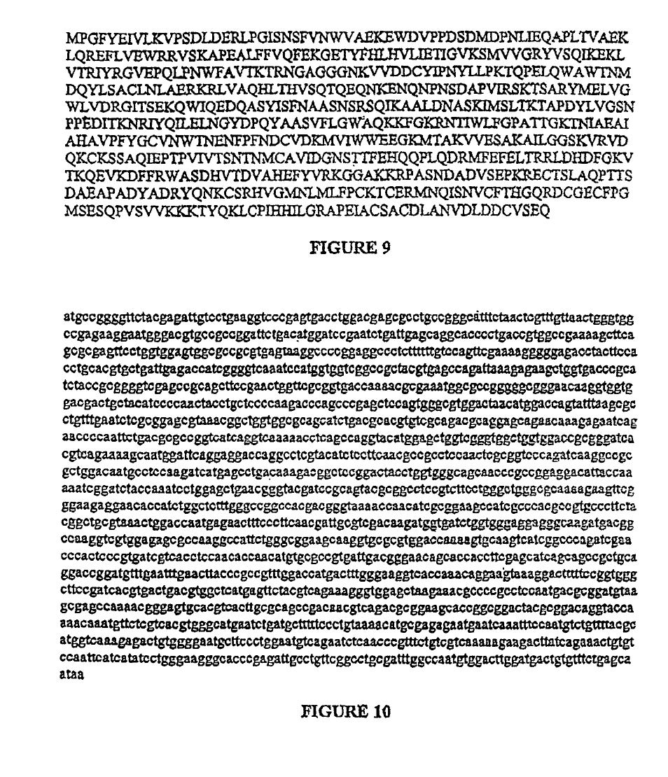

FIG. 9 depicts the amino acid sequence of a nonstructural protein isolated from adeno-associated virus 3.

FIG. 10 depicts the nucleotide sequence of a nonstructural protein isolated from adeno-associated virus 3.

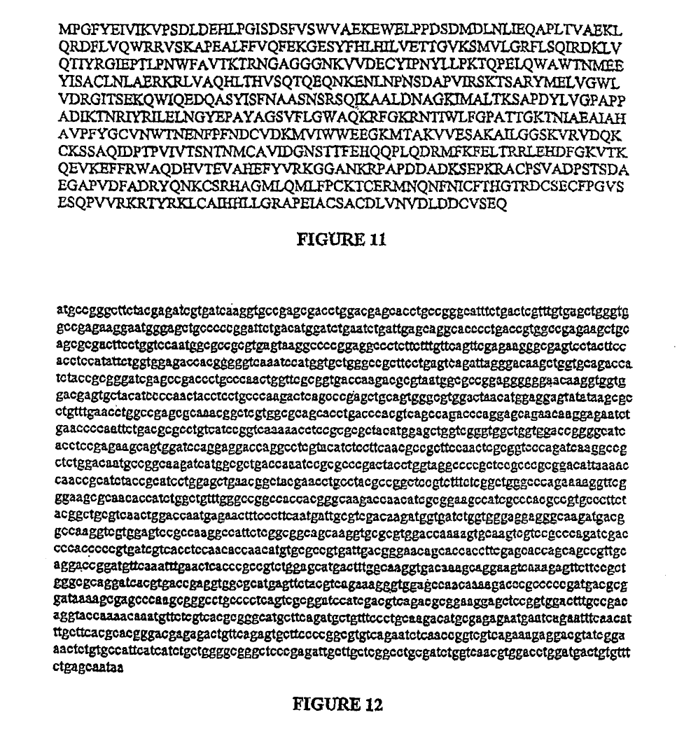

FIG. 11 depicts the amino acid sequence of a nonstructural protein isolated from adeno-associated virus 1.

FIG. 12 depicts the nucleotide sequence of a nonstructural protein isolated from adeno-associated virus 1.

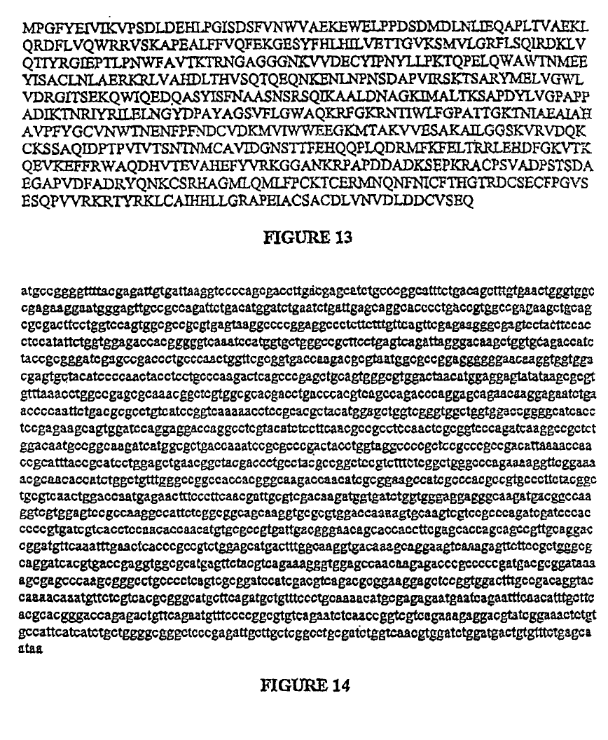

FIG. 13 depicts the amino acid sequence of Rep78 isolated from adeno-associated virus 6.

FIG. 14 depicts the nucleotide sequence of Rep78 isolated from adeno-associated virus 6.

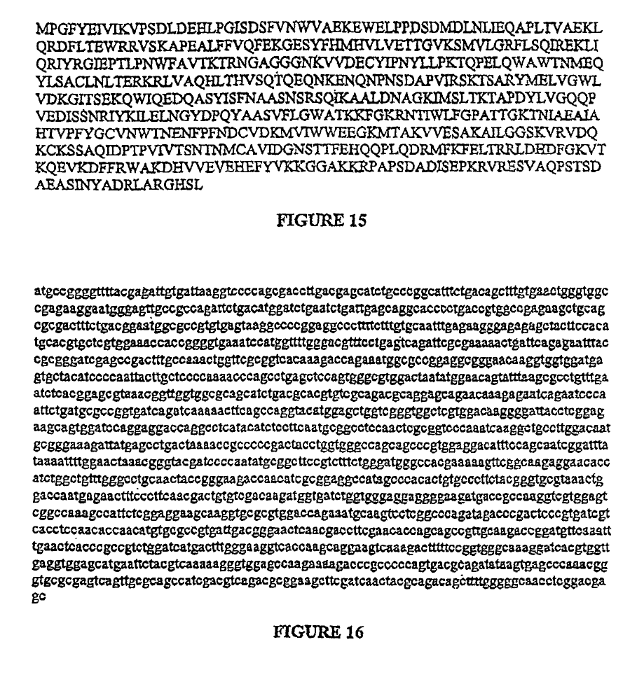

FIG. 15 depicts the amino acid sequence of Rep68 isolated from adeno-associated virus 2.

FIG. 16 depicts the nucleotide sequence of Rep68 isolated from adeno-associated virus 2.

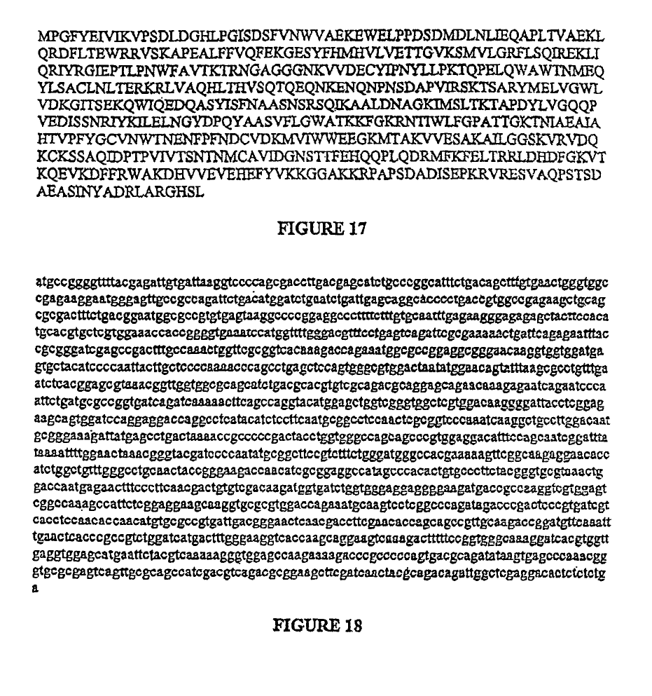

FIG. 17 depicts the amino acid sequence of major coat protein A′ (alt.) isolated from adeno-associated virus 2.

FIG. 18 depicts the nucleotide sequence of major coat protein A′ (alt.) isolated from adeno-associated virus 2.

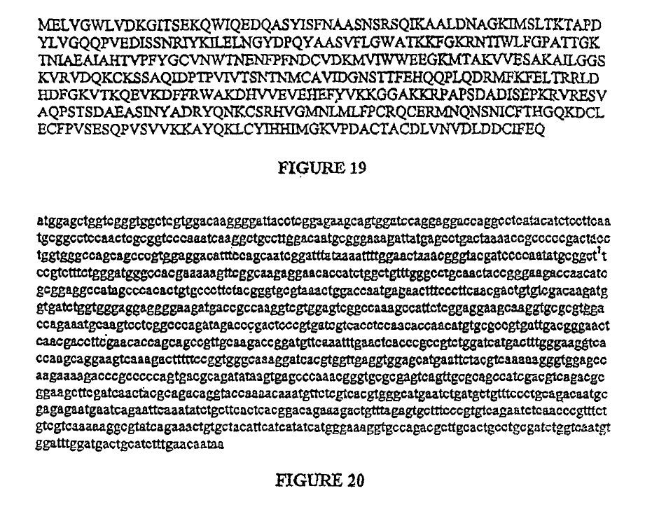

FIG. 19 depicts the amino acid sequence of major coat protein A″ (alt.) isolated from adeno-associated virus 2.

FIG. 20 depicts the nucleotide sequence of major coat protein A″ (alt.) isolated from adeno-associated virus 2.

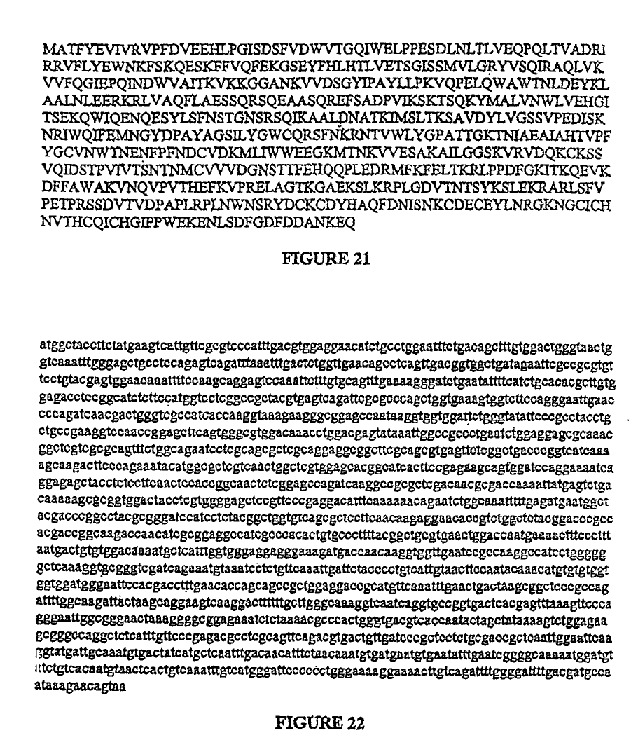

FIG. 21 depicts the amino acid sequence of a Rep protein isolated from adeno-associated virus 5.

FIG. 22 depicts the nucleotide sequence of a Rep protein isolated from adeno-associated virus 5.

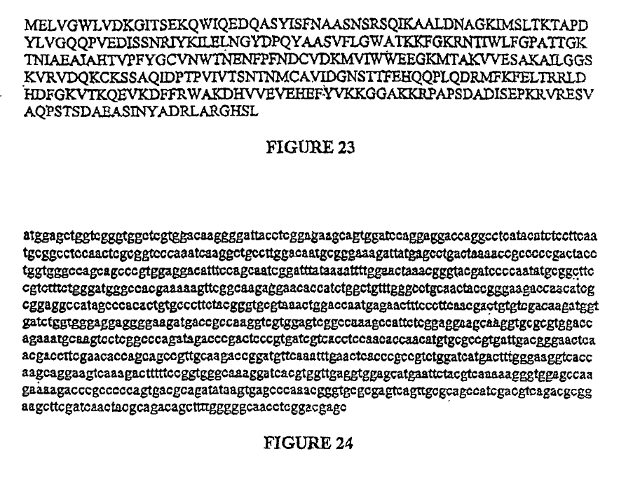

FIG. 23 depicts the amino acid sequence of major coat protein Aa (alt.) isolated from adeno-associated virus 2.

FIG. 24 depicts the nucleotide sequence of major coat protein Aa (alt.) isolated from adeno-associated virus 2.

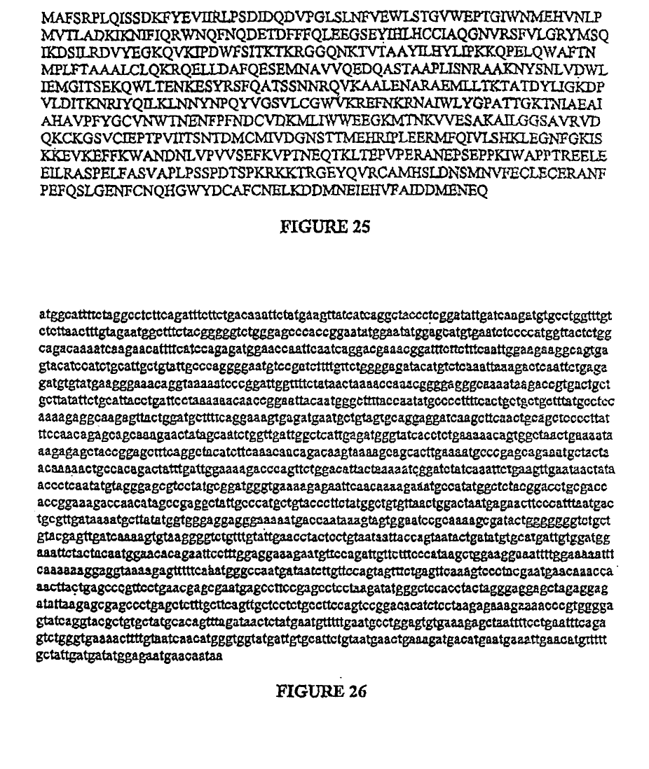

FIG. 25 depicts the amino acid sequence of a Rep protein isolated from Barbarie duck parvovirus.

FIG. 26 depicts the nucleotide sequence of a Rep protein isolated from Barbarie duck parvovirus.

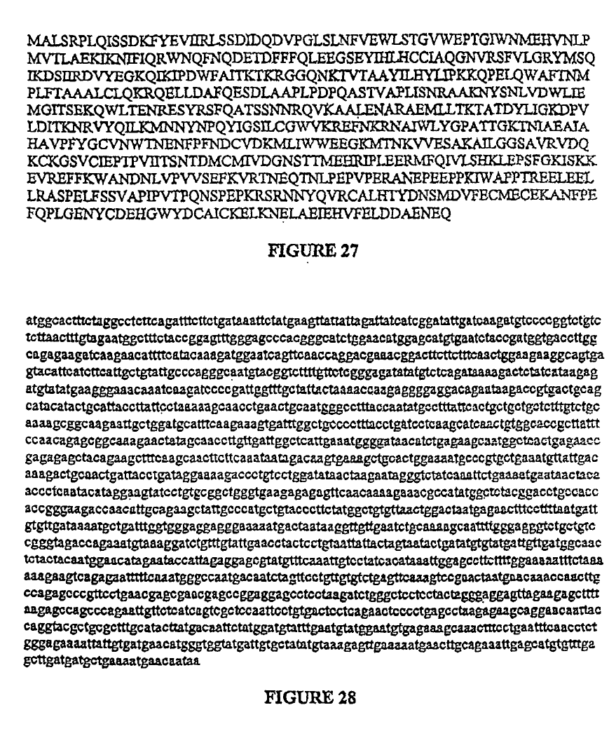

FIG. 27 depicts the amino acid sequence of a Rep protein isolated from goose parvovirus.

FIG. 28 depicts the nucleotide sequence of a Rep protein isolated from goose parvovirus.

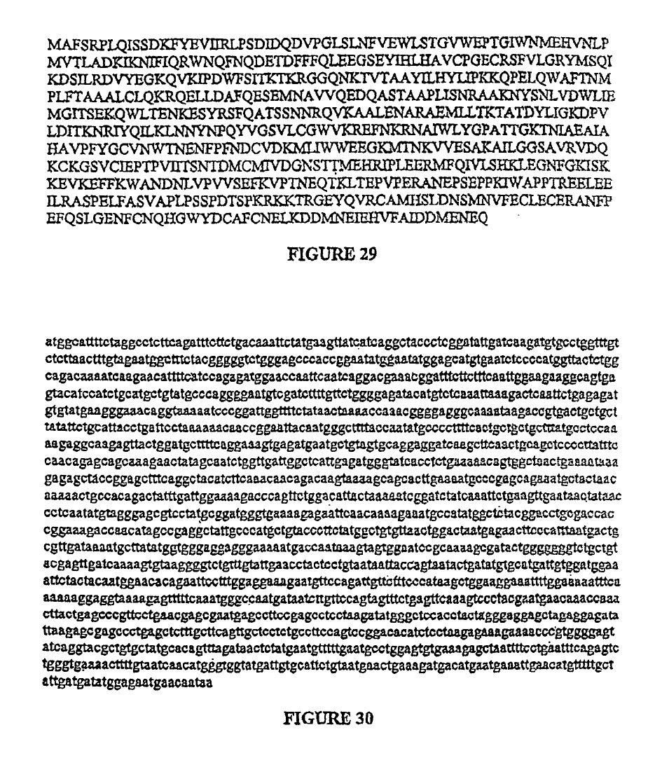

FIG. 29 depicts the amino acid sequence of NS1 isolated from muscovy duck parvovirus.

FIG. 30 depicts the nucleotide sequence of NS1 isolated from muscovy duck parvovirus.

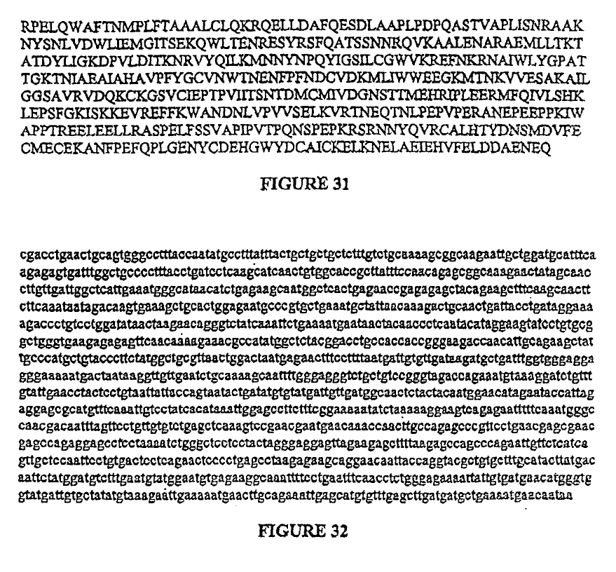

FIG. 31 depicts the amino acid sequence of NS1 isolated from goose parvovirus.

FIG. 32 depicts the nucleotide sequence of NS1 isolated from goose parvovirus.

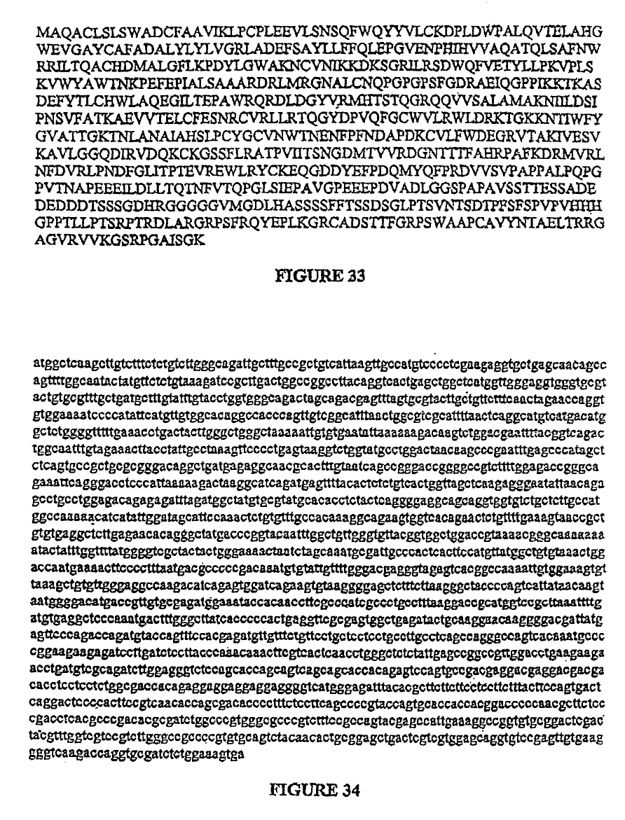

FIG. 33 depicts the amino acid sequence of non-structural protein 1 isolated from chipmunk parvovirus.

FIG. 34 depicts the nucleotide sequence of non-structural protein 1 isolated from chipmunk parvovirus.

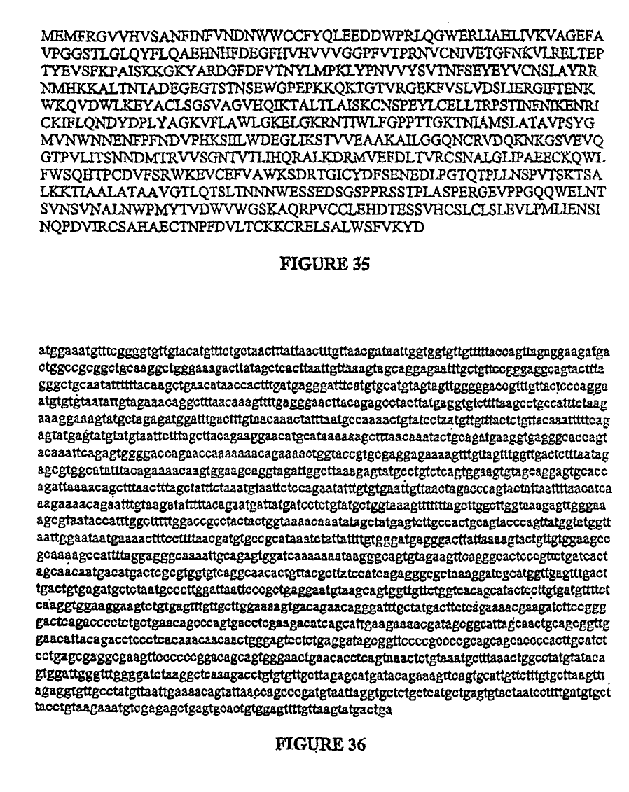

FIG. 35 depicts the amino acid sequence of non-structural protein isolated from the pig-tailed macaque parvovirus.

FIG. 36 depicts the nucleotide sequence of non-structural protein isolated from the pig-tailed macaque parvovirus.

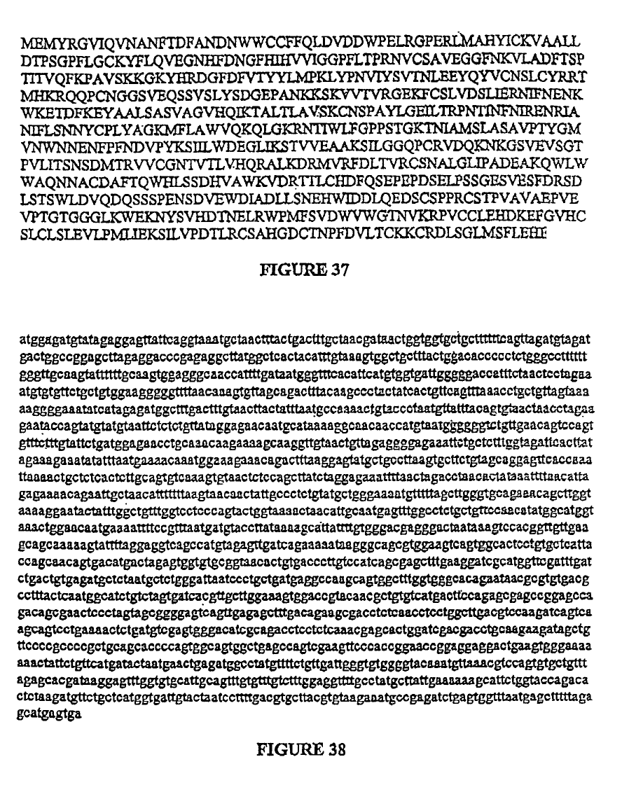

FIG. 37 depicts the amino acid sequence of NS1 isolated from a simian parvovirus.

FIG. 38 depicts the nucleotide sequence of NS1 protein isolated from a simian parvovirus.

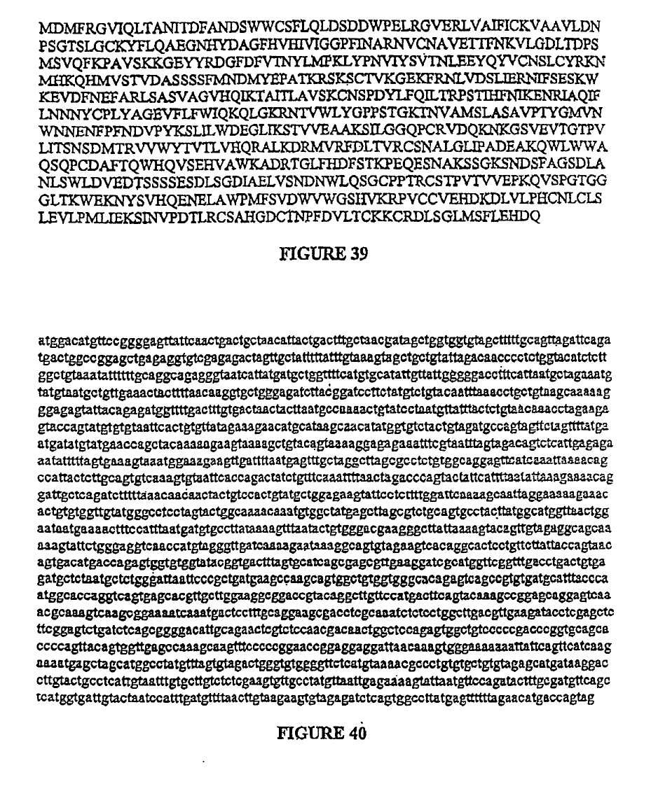

FIG. 39 depicts the amino acid sequence of a NS protein isolated from the Rhesus macaque parvovirus.

FIG. 40 depicts the nucleotide sequence of a NS protein isolated from the Rhesus macaque parvovirus.

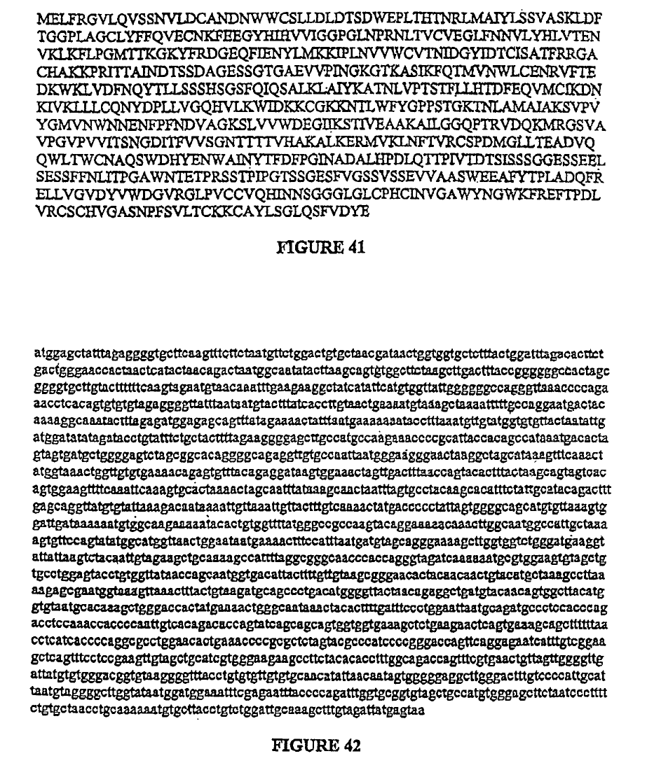

FIG. 41 depicts the amino acid sequence of a non-structural protein isolated from the B19 virus.

FIG. 42 depicts the nucleotide sequence of a non-structural protein isolated from the B19 virus.

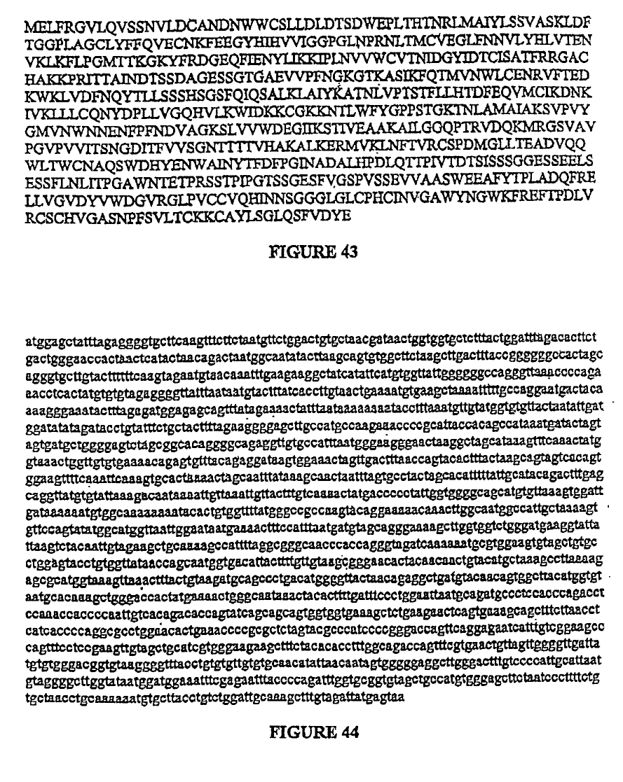

FIG. 43 depicts the amino acid sequence of r orf 1 isolated from the Erythrovirus B19.

FIG. 44 depicts the nucleotide sequence of the product of orf 1 isolated from the Erythrovirus B19.

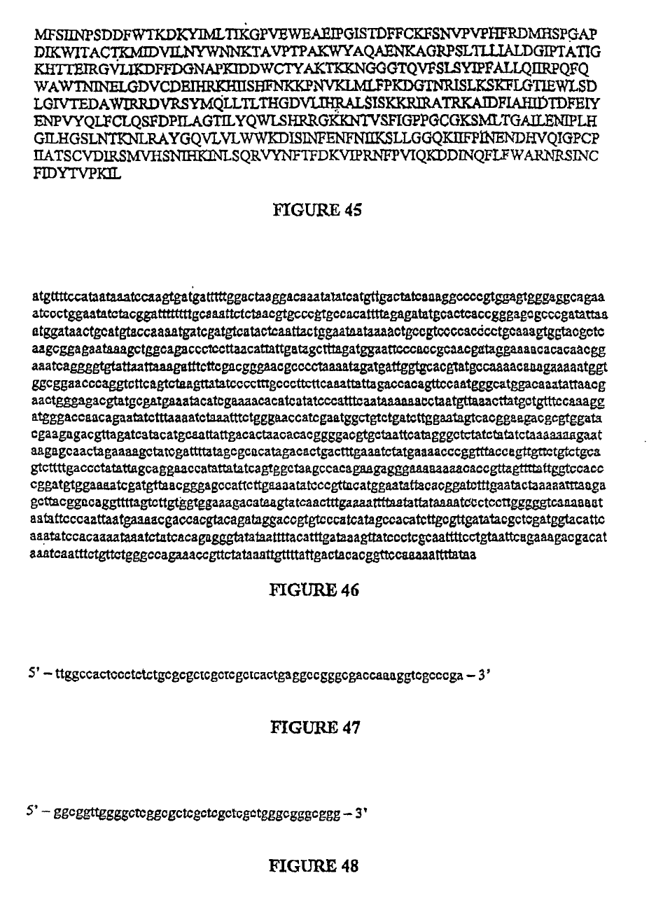

FIG. 45 depicts the amino acid sequence of U94 isolated from the human herpesvirus 6B.

FIG. 46 depicts the nucleotide sequence of U94 isolated from the human herpesvirus 6B.

FIG. 47 depicts an enzyme attachment site for a Rep protein.

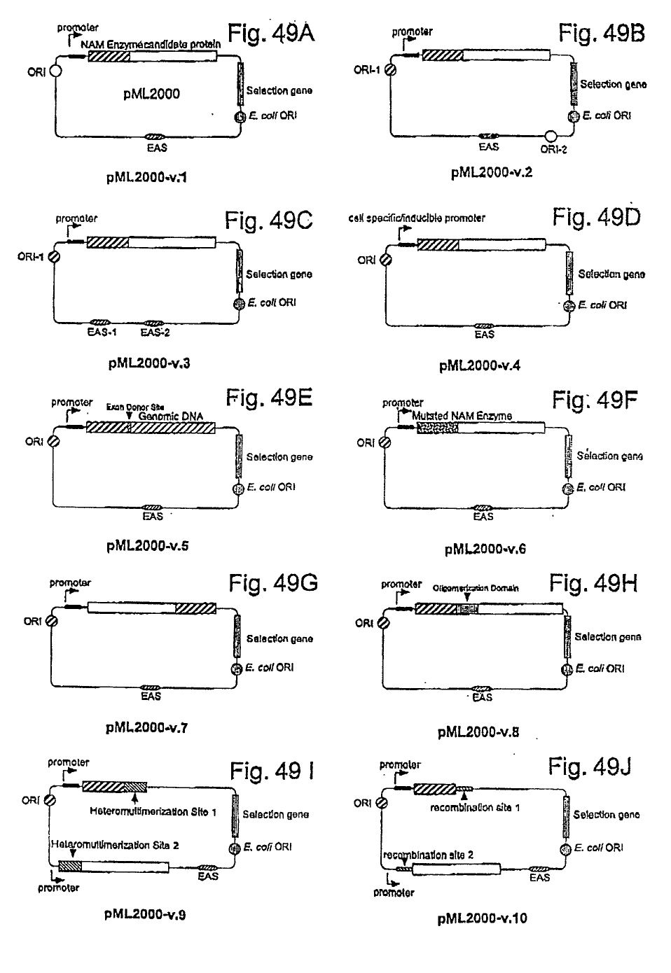

FIG. 48 depicts the Rep 68 and Rep 78 enzyme attachment site found in chromosome 19.

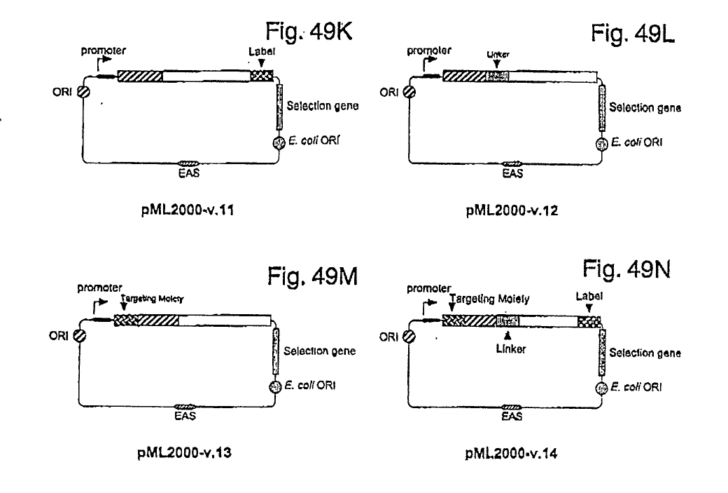

FIGS. 49A-49N depict preferred embodiments of the expression vectors of the invention.

### DETAILED DESCRIPTION

Significant effort is being channeled into screening techniques that can identify proteins relevant in signaling pathways and disease states, and to compounds that can effect these pathways and disease states. Many of these techniques rely on the screening of large libraries, comprising either synthetic or naturally occurring proteins or peptides, in assays such as binding or functional assays. One of the problems facing high throughput screening technologies today is the difficulty of elucidating the identification of the “hit”, i.e. a molecule causing the desired effect, against a background of many candidates that do not exhibit the desired properties.

The present invention is directed to a novel method that can allow the rapid and facile identification of these “hits”. The present invention relies on the use of nucleic acid modification enzymes that covalently and specifically bind to the nucleic acid molecules comprising the sequence that encodes them. Proteins of interest (for example, candidates to be screened either for binding to disease-related proteins or for a phenotypic effect) are fused (either directly or indirectly, as outlined below) to a nucleic acid modification (NAM) enzyme. The NAM enzyme will covalently attach itself to a corresponding NAM attachment sequence (termed an enzyme attachment sequence (EAS)). Thus, by using vectors that comprise coding regions for the NAM enzyme and candidate proteins and the NAM enzyme attachment sequence, the candidate protein is covalently linked to the nucleic acid that encodes it upon translation. Thus, after screening, candidates that exhibit the desired properties can be quickly isolated using a variety of methods such as PCR amplification. This facilitates the quick identification of useful candidate proteins, and allows rapid screening and validation to occur.

Accordingly, the present invention provides libraries of nucleic acid molecules comprising nucleic acid sequences encoding fusion nucleic acids encoding a nucleic acid modification enzyme and a candidate protein. By “nucleic acid” or “oligonucleotide” or grammatical equivalents herein means at least two nucleosides covalently linked together. A nucleic acid of the present invention will generally contain phosphodiester bonds, although in some cases nucleic acid analogs are included that may have alternate backbones, particularly when probes are used, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of other elements, such as labels, or to increase the stability and half-life of such molecules in physiological environments.

As will be appreciated by those in the art, all of these nucleic acid analogs may find use in the present invention. In addition, mixtures of naturally occurring nucleic acids and analogs can be made, or, alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

The present invention provides libraries of nucleic acid molecules comprising nucleic acid sequences encoding fusion nucleic acids. By “fusion nucleic acid” herein is meant a plurality of nucleic acid components (e.g., peptide coding sequences) that are joined together. The fusion nucleic acids preferably encode fusion polypeptides, although this is not required. By “fusion polypeptide” or “fusion peptide” or grammatical equivalents herein is meant a protein composed of a plurality of protein components, that while typically unjoined in their native state, are joined by their respective amino and/or carboxyl termini through a peptide linkage to form a single continuous polypeptide. Plurality in this context means at least two, and preferred embodiments generally utilize two components. It will be appreciated that the protein components can be joined directly or joined through a peptide linker/spacer as outlined below. In addition, it should be noted that in some embodiments, as is more fully outlined below, the fusion nucleic acids can encode protein components that are not fused; for example, the fusion nucleic acid may comprise an intron that is removed, leaving two non-associated protein components, although generally the nucleic acids encoding each component are fused. Furthermore, as outlined below, additional components such as fusion partners including targeting sequences, etc., can be used.

The fusion nucleic acids encode nucleic acid modification (NAM) enzymes and candidate proteins. By “nucleic acid modification enzyme” or “NAM enzyme” herein is meant an enzyme that utilizes nucleic acids, particularly DNA, as a substrate and covalently attaches itself to nucleic acid enzyme attachment (EA) sequences. The covalent attachment can be to the base, to the ribose moiety or to the phosphate moieties. NAM enzymes include, but are not limited to, helicases, topoisomerases, polymerases, gyrases, recombinases, transposases, restriction enzymes and nucleases. As outlined below, NAM enzymes include natural and non-natural variants. Although many DNA binding peptides are known, such as those involved in nucleic acid compaction, transcription regulators, and the like, enzymes that covalently attach to nucleic acids, i.e., DNA, in particular peptides involved with replication, are preferred. Some NAM enzymes can form covalent linkages with DNA without nicking the DNA. For example, it is believed that enzymes involved in DNA repair recognize and covalently attach to nucleic acid regions, which can be either double-stranded or single-stranded. Such NAM enzymes are suitable for use in the fusion enzyme library. However, DNA NAM enzymes that nick DNA to form a covalent linkage, e.g., viral replication peptides, are most preferred.

Preferably, the NAM enzyme is a protein that recognizes specific sequences or conformations of a nucleic acid substrate and performs its enzymatic activity such that a covalent complex is formed with the nucleic acid substrate. Preferably, the enzyme acts upon nucleic acids, particularly DNA, in various configurations including, but not limited to, single-strand DNA, double-strand DNA, Z-form DNA, and the like.

Suitable NAM enzymes, include, but are not limited to, enzymes involved in replication such as Rep68 and Rep78 of adeno-associated viruses (AAV), NS1 and H-1 of parvovirus, bacteriophage phi-29 terminal proteins, the 55 Kd adenovirus proteins, and derivatives thereof.

In a preferred embodiment, the NAM enzyme is a Rep protein. Rep proteins include, but are not limited to, Rep78, Rep68, and functional homologs thereof found in related viruses. Rep proteins, including their functional homologs, may be isolated from a variety of sources including parvoviruses, erythroviruse, herpesviruses, and other related viruses. One with ordinary skill in the art will appreciate that the natural Rep protein can be mutated or engineered with techniques known in the art in order to improve its activity or reduce its potential toxicity. Such experimental improvements may done in conjunction with native or variants of their corresponding EAS. One of preferred Rep proteins is the AAV Rep protein. Adeno-associated viral (AAV) Rep proteins are encoded by the left open reading frame of the viral genome. AAV Rep proteins, such as Rep68 and Rep78, regulate AAV transcription, activate AAV replication, and have been shown to inhibit transcription of heterologous promoters (Chiorini et al., J. Virol., 68(2), 797-804 (1994), hereby incorporated by reference in its entirety). The Rep68 and Rep78 proteins act, in part, by covalently attaching to the AAV inverted terminal repeat (Prasad et al., Virology, 229, 183-192 (1997); Prasad et al., Virology, 214:360 (1995); both of which are hereby incorporated by reference in their entirety). These Rep proteins act by a site-specific and strand-specific endonuclease nick at the AAV origin at the terminal resolution site, followed by covalent attachment to the 5′ terminus of the nicked site via a putative tyrosine linkage. Rep68 and Rep78 result from alternate splicing of the transcript. The nucleic acid sequence of Rep68 is shown in FIG. 15, and the protein sequence in FIG. 16; the nucleic acid and protein sequences of Rep78 proteins isolated from various sources are shown in FIGS. 1, 2, 7, 8, 13, and 14. As is further outlined below, functional fragments, variants, and homologs of Rep proteins are also included within the definition of Rep proteins; in this case, the variants preferably include nucleic acid binding activity and endonuclease activity. The corresponding enzyme attachment site for Rep68 and Rep78, discussed below, is shown in FIGS. 47 and 48 and is set forth in Example 1.

In a preferred embodiment, the NAM enzyme is NS1. NS1 is a non-structural protein in parvovirus, is a functional homolog of Rep78, and also covalently attaches to DNA (Cotmore et al., J. Virol., 62(3), 851-860 (1998), hereby expressly incorporated by reference). The nucleotide and amino acid sequences of NS1 proteins isolated from various sources are shown in FIGS. 9-12, 29-34, 37, and 38. As is further outlined below, fragments and variants of NS1 proteins are also included within the definition of NS1 proteins.

In a preferred embodiment, the NAM enzyme is the parvoviral H-1 protein, which is also known to form a covalent linkage with DNA (see, for example, Tseng et al., Proc. Natl. Acad. Sci. USA, 76(11), 5539-5543 (1979), hereby expressly incorporated by reference. As is further outlined below, fragments and variants of H-1 proteins are also included within the definition of H-1 proteins.

In a preferred embodiment, the NAM enzyme is the bacteriophage phi-29 terminal protein, which is also known to form a covalent linkage with DNA (see, for example, Germendia et al., Nucleic Acid Research, 16(3), 5727-5740 (1988), hereby expressly incorporated by reference). As is further outlined below, fragments and variants of phi-29 proteins are also included within the definition of phi-29 proteins.

The NAM enzyme also can be the adenoviral 55 Kd (a55) protein, again known to form covalent linkages with DNA; see Desiderio and Kelly, J. Mol. Biol., 98, 319-337 (1981), hereby expressly incorporated by reference. As is further outlined below, fragments and variants of a55 proteins are also included within the definition of a55 proteins.

The nucleic acid sequences and amino acid sequences of other Rep homologs that are suitable for use as NAM enzymes are set forth in FIGS. 3-6, 17-28, 35, 36, and 39-46.

Some DNA-binding enzymes form covalent linkages upon physical or chemical stimuli such as, for example, UV-induced crosslinking between DNA and a bound protein, or camptothecin (CPT)related chemically induced trapping of the DNA-topoisomerase I covalent complex (e.g., Hertzberg et al., J. Biol. Chem., 265, 19287-19295 (1990)). NAM enzymes that form induced covalent linkages are suitable for use in some embodiments of the present invention.

Also included with the definition of NAM enzymes of the present invention are amino acid sequence variants retaining biological activity (e.g., the ability to covalently attach to nucleic acid molecules). These variants fall into one or more of three classes: substitutional, insertional or deletional (e.g. fragment) variants. These variants ordinarily are prepared by site specific mutagenesis of nucleotides in the DNA encoding the NAM protein, using cassette or PCR mutagenesis or other techniques well known in the art, to produce DNA encoding the variant, and thereafter expressing the recombinant DNA in cell culture as outlined herein. However, variant NAM protein fragments having up to about 100-150 residues may be prepared by in vitro synthesis or peptide ligation using established techniques. Amino acid sequence variants are characterized by the predetermined nature of the variation, a feature that sets them apart from naturally occurring allelic or interspecies variation of the NAM protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed NAM variants screened for the optimal combination of desired activity. Techniques for making substitution mutations at predetermined sites in DNA having a known sequence are well known, for example, M13 primer mutagenesis and PCR mutagenesis. Screening of the mutants, variants, homologs, etc., is accomplished using assays of NAM protein activities employing routine methods such as, for example, binding assays, affinity assays, peptide conformation mapping, and the like.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger, for example when unnecessary domains are removed.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the NAM protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | lu |
| Cys | Ser |
| Gln | Asn |
| Glu | Asp |
| Gly | Pro |
| His | Asn, Gin |
| Ile | Leu, Val |
| Leu | Ile, Val |
| Lys | Arg, Gln, Glu |
| Met | Leu, Ile |
| PheSer | Met, Leu, Tyr |
| Thr | Thr |
| Trp | Ser |
| Tyr | Tyr |
| Val | Trp, Phe |
| | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. The variants typically exhibit the same qualitative biological activity as the naturally-occurring analogue, although variants also are selected to modify the characteristics of the NAM proteins as needed. Alternatively, the variant may be designed such that the biological activity of the NAM protein is altered. For example, glycosylation sites may be altered or removed. Similarly, functional mutations within the endonuclease domain or nucleic acid recognition site may be made. Furthermore, unnecessary domains may be deleted, to form fragments of NAM enzymes.

In addition, some embodiments utilize concatameric constructs to effect multivalency and increase binding kinetics or efficiency. For example, constructs containing a plurality of NAM coding regions or a plurality of EASs may be made.

Also included with the definition of NAM protein are other NAM homologs, and NAM proteins from other organisms including viruses, which are cloned and expressed as known in the art. Thus, probe or degenerate polymerase chain reaction (PCR) primer sequences may be used to find other related NAM proteins. As will be appreciated by those in the art, particularly useful probe and/or PCR primer sequences include the unique areas of the NAM nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition to nucleic acids encoding NAM enzymes, the fusion nucleic acids of the invention also encode candidate proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures, the latter being especially useful when the target molecule is a protein. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard ex vivo degradations. Chemical blocking groups or other chemical substituents may also be added. Thus, the present invention can find use in template based synthetic systems.

By “candidate protein” herein is meant a protein to be tested for binding, association or effect in an assay of the invention, including both in vitro (e.g. cell free systems) or ex vivo (within cells). The candidate peptide comprises at least one desired target property. The desired target property will depend upon the particular embodiment of the present invention. “Target property” refers to an activity of interest. Optionally, the target property is used directly or indirectly to identify a subset of fusion protein-expression vector conjugates, thus allowing for the retrieval of the desired NAP conjugates from the fusion protein library. Target properties include, for example, the ability of the encoded display peptide to mediate binding to a partner, enzymatic activity, the ability to mimic a given factor, the ability to alter cell physiology, and structural or other physical properties including, but not limited to, electromagnetic behavior or spectroscopic behavior of the peptides. Generally, as outlined below, libraries of candidate proteins are used in the fusions. As will be appreciated by those in the art, the source of the candidate protein libraries can vary, particularly depending on the end use of the system.

In a preferred embodiment, the candidate proteins are derived from cDNA libraries. The cDNA libraries can be derived from any number of different cells, particularly those outlined for host cells herein, and include cDNA libraries generated from eucaryotic and procaryotic cells, viruses, cells infected with viruses or other pathogens, genetically altered cells, etc. Preferred embodiments, as outlined below, include cDNA libraries made from different individuals, such as different patients, particularly human patients. The cDNA libraries may be complete libraries or partial libraries.

Furthermore, the library of candidate proteins can be derived from a single cDNA source or multiple sources; that is, cDNA from multiple cell types or multiple individuals or multiple pathogens can be combined in a screen. The cDNA library may utilize entire cDNA constructs or fractionated constructs, including random or targeted fractionation. Suitable fractionation techniques include enzymatic, chemical or mechanical fractionation.

In a preferred embodiment, the candidate proteins are derived from genomic libraries. As above, the genomic libraries can be derived from any number of different cells, particularly those outlined for host cells herein, and include genomic libraries generated from eucaryotic and procaryotic cells, viruses, cells infected with viruses or other pathogens, genetically altered cells, etc. Preferred embodiments, as outlined below, include genomic libraries made from different individuals, such as different patients, particularly human patients. The genomic libraries may be complete libraries or partial libraries. Furthermore, the library of candidate proteins can be derived from a single genomic source or multiple sources; that is, genomic DNA from multiple cell types or multiple individuals or multiple pathogens can be combined in a screen. The genomic library may utilize entire genomic constructs or fractionated constructs, including random or targeted fractionation. Suitable fractionation techniques include enzymatic, chemical or mechanical fractionation.

In this regard, the combination of a NAM enzyme with nucleic acid derived from genomic DNA in a genetic library vector is novel. Accordingly, the present invention further provides an isolated and purified nucleic acid molecule comprising a nucleic acid sequence encoding a NAM enzyme fused to a nucleic acid sequence isolated or derived from genomic DNA (for example, vectors comprising genomic digests can be made, or specific genomic sequences can be amplified and/or purified and the amplicons used). Such an isolated and purified nucleic acid molecule is particularly useful in the present inventive methods described herein. Preferably, the isolated and purified nucleic acid molecule further comprises a splice donor sequence or splice acceptor sequence located between the nucleic acid sequence encoding the NAM enzyme and the genomic DNA. The incorporation of splice donor and/or splice acceptor sequences into the isolated and purified nucleic acid sequence allows formation of a transcript encoding the NAM enzyme and exons of the genomic DNA fragment. The methods of the prior art have failed to comprehend the potential of operably linking genomic DNA to a NAM enzyme such that the product of the genomic DNA can be associated with the nucleic acid molecule encoding it. One of ordinary skill in the art will appreciate that appropriate regulatory sequences can also be incorporated into the isolated and purified nucleic acid molecule.

In a preferred embodiment, the present invention also provides methods of determining open reading frames in genomic DNA. In this embodiment, the candidate protein encoded by the genomic nucleic acid is preferably fused directly to the N-terminus of the NAM enzyme, rather than at the C-terminus. Thus, if a functional NAM enzyme is produced, the genomic DNA was fused in the correct reading frame. This is particularly useful with the use of labels, as well.

In addition, the libraries may also be subsequently mutated using known techniques (exposure to mutagens, error-prone PCR, error-prone transcription, combinatorial splicing (e.g. cre-lox recombination)). In this way libraries of procaryotic and eukaryotic proteins may be made for screening in the systems described herein. Particularly preferred in this embodiment are libraries of bacterial, fungal, viral, plant, and animal (e.g., mammalian) proteins, with the latter being preferred, and human proteins being especially preferred.

The candidate proteins may vary in size. In the case of cDNA or genomic libraries, the proteins may range from 20 or 30 amino acids to thousands, with from about 50 to 1000 (e.g., 75, 150, 350, 750 or more) being preferred and from 100 to 500 (e.g., 200, 300, or 400) being especially preferred. When the candidate proteins are peptides, the peptides are from about 3 to about 50 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In a preferred embodiment, libraries of candidate proteins are fused to the NAM enzymes, with each member of the library comprising a different candidate protein. However, as will be appreciated by those in the art, different members of the library may be reproduced or duplicated, resulting in some libraries members being identical. The library should provide a sufficiently structurally diverse population of expression products to effect a probabilistically sufficient range of cellular responses to provide one or more cells exhibiting a desired response. Accordingly, an interaction library must be large enough so that at least one of its members will have a structure that gives it affinity for some molecule, including both protein and non-protein targets, or other factors whose activity is necessary or effective within the assay of interest. Although it can be difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length has the potential to code for 20⁷ (10⁹) to 20²⁰. Thus, with libraries of 10⁷ to 10⁸ the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different expression products are simultaneously analyzed in the subject methods, although libraries of less complexity (e.g., 10², 10³, 10⁴, or 10⁵ different expression products) or greater complexity (e.g., 10¹⁰, 10¹¹, or 10¹² different expression products) are appropriate for use in the present invention. Preferred methods maximize library size and diversity.

In any library system encoded by oligonucleotide synthesis, complete control over the codons that will eventually be incorporated into the peptide structure is difficult. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is a high likelihood that 46.7% of the peptides will prematurely terminate. One way to alleviate this is to have random residues encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 27% chance to terminate prematurely. Alternatively, fusing the candidate proteins to the C-terminus of the NAM enzyme also may be done, although in some instances, fusing to the N-terminus means that prematurely terminating proteins result in a lack of NAM enzyme which eliminates these samples from the assay.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, PDZ domains, serines, threonines, tyrosines or histidines for phosphorylation sites, etc., or to purines, etc.

In a preferred embodiment, the bias is towards peptides or nucleic acids that interact with known classes of molecules. For example, when the candidate protein is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate proteins as well.

Thus, a number of molecules or protein domains are suitable as starting points for the generation of biased randomized candidate proteins. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

In a preferred embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases:

1. XXXPPXPXX, wherein X is a randomized residue.

2. (within the positions of residue positions 11 to -2):

| | | | | | | | | | | | | | | | | | |
|---|---|---|---|---|---|---|---|---|---|---|---|---|---|---|---|---|---|
| | | 11 | 10 | 9 | 8 | 7 | 6 | 5 | 4 | 3 | 2 | 1 | | | | | |
| Met | Gly | aa11 | aa10 | aa9 | aa8 | aa7 | Arg | Pro | Leu | Pro | Pro | hyd | | | | | |
| 0 | −1 | −2 | | | | | | | | | | | | | | | |
| Pro | hyd | hyd | Gly | Gly | Pro | Pro | STOP | | | | | | | | | | |
| atg | ggc | nnk | nnk | nnk | nnk | nnk | aga | cct | ctg | cct | cca | sbk | ggg | sbk | sbk | gga | ggc cca cct TAA1. |

In this embodiment, the N-terminus flanking region is suggested to have the greatest effects on binding affinity and is therefore entirely randomized. “Hyd” indicates a bias toward a hydrophobic residue, i.e.-Val, Ala, Gly, Leu, Pro, Arg. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

Thus, in a preferred embodiment, the candidate protein is a structural tag that will allow the isolation of target proteins with that structure. That is, in the case of leucine zippers, the fusion of the NAM enzyme to a leucine zipper sequence will allow the fusions to “zip up” with other leucine zippers, allow the quick isolation of a plurality of leucine zipper proteins.

In addition, structural tags (which may only be the proteins themselves) can allow heteromultimeric protein complexes to form, that then are assayed for activity as complexes. That is, many proteins, such as many eucaryotic transcription factors, function as heteromultimeric complexes which can be assayed using the present invention.

In addition, rather than a cDNA, genomic, or random library, the candidate protein library may be a constructed library; that is, it may be built to contain only members of a defined class, or combinations of classes. For example, libraries of immunoglobulins may be built, or libraries of G-protein coupled receptors, tumor suppressor genes, proteases, transcription factors, phosphatases, kinases, etc.

The fusion nucleic acid can comprise the NAM enzyme and candidate protein in a variety of configurations, including both direct and indirect fusions, and include N- and C-terminal fusions and internal fusions.

In a preferred embodiment, the NAM enzyme and the candidate protein are directly fused. In this embodiment, a direct, in-frame fusion of the nucleic acid encoding the NAM enzyme and the candidate protein is engineered. The library of fusion peptides can be constructed as N- and/or C-terminal fusions and internal fusions. Thus, the NAM enzyme coding region may be 3′ or 5′ to the candidate protein coding region, or the candidate protein coding region may be inserted into a suitable position within the coding region of the NAM enzyme. In this embodiment, it may be desirable to insert the candidate protein into an external loop of the NAM enzyme, either as a direct insertion or with the replacement of several of the NAM enzyme residues. This may be particularly desirable in the case of random candidate proteins, as they frequently require some sort of scaffold or presentation structure to confer a conformationally restricted structure. For an example of this general idea using green fluorescent protein (GFP) as a scaffold for the expression of random peptide libraries, see for example WO 99/20574, expressly incorporated herein by reference.

In a preferred embodiment, the NAM enzyme and the candidate protein are indirectly fused. This may be accomplished such that the components of the fusion remain attached, such as through the use of linkers, in ways that result in the components of the fusion becoming separated after translation, or, alternatively, in ways that start with the NAM enzyme and the candidate protein being made separately and then joined.

In a preferred embodiment, linkers may be used to functionally isolate the NAM enzyme and the candidate protein. That is, a direct fusion system may sterically or functionally hinder the interaction of the candidate protein with its intended binding partner, and thus fusion configurations that allow greater degrees of freedom are useful. An analogy is seen in the single chain antibody area, where the incorporation of a linker allows functionality. As will be appreciated by those in the art, there are a wide variety of different types of linkers that may be used, including cleavable and non-cleavable linkers; this cleavage may also occur at the level of the nucleic acid, or at the protein level.

In a preferred embodiment, linkers known to confer flexibility are used. For example, useful linkers include glycine-serine polymers (including, for example, (GS)ₙ, and (GGGS)ₙ, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

The linker used to construct indirect fusion enzymes can be a cleavable linker. Cleavable linkers can function at the level of the nucleic acid or the protein. That is, cleavage (which in this sense means that the NAM enzyme and the candidate protein are separated) can occur during transcription, or before or after translation.

With respect to cleavable linkers, the cleavage can occur as a result of a cleavage functionality built into the nucleic acid. In this embodiment, for example, cleavable nucleic acid sequences, or sequences that will disrupt the nucleic acid, can be used. For example, intron sequences that the cell will remove can be placed between the coding region of the NAM enzyme and the candidate protein. In a preferred embodiment, the linkers are heterodimerization domains. In this embodiment, both the NAM enzyme and the candidate protein are fused to heterodimerization domains (or multimeric domains, if multivalency is desired), to allow association of these two proteins after translation.

In a preferred embodiment, cleavable protein linkers are used. In this embodiment, the fusion nucleic acids include coding sequences for a protein sequence that may be subsequently cleaved, generally by a protease. As will be appreciated by those in the art, cleavage sites directed to ubiquitous proteases, e.g. those that are constitutively present in most or all of the host cells of the system, can be used. Alternatively, cleavage sites that correspond to cell-specific proteases may be used. Similarly, cleavage sites for proteases that are induced only during certain cell cycles or phases or are signal specific events may be used as well.

There are a wide variety of possible proteinaceous cleavage sites known. For example, sequences that are recognized and cleaved by a protease or cleaved after exposure to certain chemicals are considered cleavable linkers. This may find particular use in in vitro systems, outlined below, as exogeneous enzymes can be added to the milieu or the NAP conjugates may be purified and the cleavage agents added. For example, cleavable linkers include, but are not limited to, the prosequence of bovine chymosin, the prosequence of subtilisin, the 2a site (Ryan et al., J. Gen. Virol. 72:2727 (1991); Ryan et al., EMBO J. 13:928 (1994); Donnelly et al., J. Gen. Virol. 78:13 (1997); Hellen et al., Biochem, 28(26):9881 (1989); and Mattion et al., J. Virol. 70:8124 (1996)), prosequences of retroviral proteases including human immunodeficiency virus protease and sequences recognized and cleaved by trypsin (EP 578472, Takasuga et al., J. Biochem. 112(5)652 (1992)) factor Xa (Gardella et al., J. Biol. Chem. 265(26):15854 (1990), WO 9006370), collagenase (J03280893, Tajima et al., J. Ferment. Bioeng. 72(5):362 (1991), WO 9006370), clostripain (EP 578472), subtilisin (including mutant H64A subtilisin, Forsberg et al., J. Protein Chem. 10(5):517 (1991), chymosin, yeast KEX2 protease (Bourbonnais et al., J. Bio. Chem. 263(30):15342 (1988), thrombin (Forsberg et al., supra; Abath et al., BioTechniques 10(2):178 (1991)), Staphylococcus aureus V8 protease or similar endoproteinase-Glu-C to cleave after Glu residues (EP 578472, Ishizaki et al., Appl. Microbiol. Biotechnol. 36(4):483 (1992)), cleavage by Nla proteainase of tobacco etch virus (Parks et al., Anal. Biochem. 216(2):413 (1994)), endoproteinase-Lys-C (U.S. Pat. No. 4,414,332) and endoproteinase-Asp-N, Neisseria type 2 IgA protease (Pohlner et al., Bio/Technology 10(7):799-804 (1992)), soluble yeast endoproteinase yscF (EP 467839), chymotrypsin (Altman et al., Protein Eng. 4(5):593 (1991)), enteropeptidase (WO 9006370), Iysostaphin, a polyglycine specific endoproteinase (EP 316748), and the like. See e.g. Marston, F.A.O. (1986) Biol. Chem. J. 240, 1-12. Particular amino acid sites that serve as chemical cleavage sites include, but are not limited to, methionine for cleavage by cyanogen bromide (Shen, PNAS USA 81:4627 (1984); Kempe et al., Gene 39:239 (1985); Kuliopulos et al., J. Am. Chem. Soc. 116:4599 (1994); Moks et al., Bio/Technology 5:379 (1987); Ray et al., Bio/Technology 11:64 (1993)), acid cleavage of an Asp-Pro bond (Wingender et al., J. Biol. Chem. 264(8):4367 (1989); Gram et al., Bio/Technology 12:1017 (1994)), and hydroxylamine cleavage at an Asn-Gly bond (Moks, supra).

In addition, there are a variety of additional fusion techniques that can be used, including a variety of pre- and post-translational fusion techniques, as outlined below. That is, the NAM enzyme and the candidate protein can be made separately and then joined later. Similarly, the nucleic acids encoding these components can be made separately and joined later as well.

Accordingly, the nucleic acids of the present invention can be expressed as cis-fusions and as trans-fusions. As described above, when the nucleic acids of the present invention are expressed as cis-fusions, the expressed protein contains both the NAM enzyme (e.g. the Rep protein) and the candidate protein. Thus, a fusion polypeptide is formed via transcription of a single messenger RNA.

The nucleic acids of the present invention also can be expressed as trans-fusions. In this embodiment, the NAM enzyme and the candidate protein are expressed separately as fusions with one or more merger moieties that allow later fusion; for example, a merger moiety can have the ability to participate in a ligation reaction, or have the ability to participate in a cross-linking reaction. The resulting fusions are then joined to form a fusion protein in which the NAM enzyme is generally (but not required to be) covalently linked to the candidate protein.

Suitable ligation reactions include, but are not limited to, the ligation reaction mediated by ubiquitin protein ligase, and an intein catalyzed trans-ligation reaction. A suitable cross-linking reaction is the cross-linking reaction catalyzed by transglutaminase.

In a preferred embodiment, the ligation reaction is mediated by ubiquitin protein ligase. The ubiquitin protein ligase is one component of the ubiquitin pathway (Ciechanover and Schwartz, (1998) Proc. Natl. Acad. Sci., USA, 95:2727-2730). The ubiquitin pathway consists of several components that act in concert. Of these components, those of interest for the present invention are components that participate in the covalent attachment of ubiquitin molecules to a protein substrate. Briefly, the covalent attachment of ubiquitin to a protein occurs as follows. Ubiquitin, an evolutionarily conserved protein of 76 residues, is activated in its C-terminal glycine to a high energy thiol ester intermediate, a reaction catalyzed by the ubiquitin-activating enzyme, El. After activation, one of several E2 enzymes (ubiquitin-carrier proteins or ubiquitin-conjugating enzymes, UBCs) transfers the activated ubiquitin moiety from El to a member of the ubiquitin protein ligase family, E3, to which the substrate protein is specifically bound. E3 catalyzes the last step in the conjugation process, covalent attachment of ubiquitin to the substrate. A polyubiquitin chain may be formed by the transfer of additional activated moieties to lysine⁴⁸ of the previously conjugated ubiquitin molecule. After conjugation, the ubiquitinylated protein may be targeted for degradation by the proteasome. However, ubiquitin modification is not limited to targeting of proteins for degradation, thus not all ubiquitinylated proteins are targeted for degradation (Ciechanover and Schwartz, (1998) Proc. Natl. Acad. Sci., USA, 95:2727-2730).

In a preferred embodiment, the nucleic acid encoding a NAM enzyme is covalently attached to a nucleic acid encoding a ligation mediating moiety to form a first fusion nucleic acid. By “ligation mediating moiety” herein is meant an enzyme that is capable of modifying a substrate such that the substrate is able to participate in a ligation reaction. Preferably, the ligation mediating moiety is the ubiquitin activating enzyme, El, but other enzymes with similar properties may also be used (see Ciechanover and Schwartz, (1998) Proc. Natl. Acad. Sci., USA, 95:2727-2730).

In a preferred embodiment, the nucleic acid encoding a candidate protein is covalently attached to a nucleic acid encoding a ligation substrate to form a second fusion nucleic acid. By “ligation substrate” herein is meant a substrate that can be modified by an enzyme, such that the modified substrate can participate in a ligation reaction. Preferably, the ligation substrate is ubiquitin (from any species), but other substrates with similar properties may also be used (see Ciechanover and Schwartz, (1998) Proc. Natl. Acad. Sci., USA, 95:2727-2730) Unless specified, the use of the terms “first” and “second” are not meant to imply any order or hierarchy.

Once made, the fusion nucleic acids are combined either in vitro or in vivo such that E1 activation of ubiquitin occurs. Activation of ubiquitin results in the formation of a covalent linkage between the E1-NAM enzyme fusion and the ubiquitin-candidate fusion, thereby creating a fusion polypeptide comprising a NAM enzyme and a candidate protein.

As will be appreciated by those of skill in the art, fusion nucleic acids may be made in which the NAM enzyme is fused to ubiquitin and the candidate protein is fused to E1.

Other embodiments include the creation of fusion nucleic acids wherein either the NAM enzyme or the candidate protein is engineered to have multiple ubiquitination sites. For example, if the NAM enzyme has mulitple ubiquitination sites, the ubiquitin-candidate protein will be linked to the ε-NH₂ of the lysine residue in the modified NAM enzyme.

In a preferred embodiment, the ligation reaction is an intein catalyzed trans-ligation reaction. Inteins are self-splicing proteins that occur as in-frame insertions in specific host proteins. In a self-splicing reaction, inteins excise themselves from a precursor protein, while the flanking regions, the exteins, become joined via a new peptide bond to form a linear protein.

Many inteins, are bifunctional proteins mediating both protein splicing and DNA cleavage. Such elements consist of a protein splicing domain interrupted by an endonuclease domain. Because endonuclease activity is not required for protein splicing, mini-inteins, with accurate splicing activity can be generated by deletion of this central domain (Wood, et al., (1999) Nature Biotechnology, 17:889-892).

Protein splicing involves four nucleophilic displacements by three conserved splice junction residues. These residues, located near the intein/extein junctions, include the initial cysteine, serine, or threonine of the intein, which intiates splicing with an acyl shift. The conserved cysteine, serine, or threonine of the extein, which ligates the exteins through nucleophilic attack, and the conserved C-terminal histidine and asparagine of the intein, which releases the intein from the ligated exteins through succinimide formation. See Wood, et al., (1999) supra.

Inteins also catalyze a trans-ligation reaction The ability of intein function to be reconstituted in trans by spatially separated intein domains suggests that the self-splicing motifs or mini inteins can be used to link any two peptides or polypeptides that are fused to the mini-inteins (Mills, et al., (1998) Proc. Natl. Acad. Sci., USA, 95:3543-3548).

By “inteins”, or “mini-inteins” or “intein motifs”, or “intein domains”, or grammatical equivalents herein is meant a protein sequence which, during protein splicing, is excised from a protein precursor.

In a preferred embodiment, the NAM enzyme fusion nucleic acid is designed with the primary sequence from the N-terminus of a suitable intein; thus the fusion nucleic acid comprise I_{N}-NAM enzyme. I_{N} is defined herein as the N-terminal intein motif and the NAM enzyme is defined as described herein. The candidate protein fusion nucleic acid is designed with the primary sequence from the C-terminus of a suitable intein; thus the fusion nucleic acid comprises I_{C}-candidate protein. I_{C} is defined herein as the C-terminal intein motif and the candidate protein is defined as described above. DNA sequences encoding the inteins may be obtained from a prokaryotic DNA sequence, such as a bacterial DNA sequence, or a eukaryotic DNA sequence, such as a yeast DNA sequence.

The Intein Registry includes a list of all experimental and theoretical inteins discovered to date and submitted to the registry (http)://www.neb.com/inteins/int reg.html).

In a preferred embodiment, fusion polypeptides are designed using intein motifs selected from organisms belonging to the Eucarya and Eubacteria, with the intein Ssp DnaB (GenBank accession number Q55418) being particularly preferred. The GenBank accession numbers for other intein proteins and nucleic acids include, but are not limited to: Ceu ClpP (GenBank acession number P42379); CIV RIR1 (T03053); Ctr VMA (GenBank accession number A46080); Gth DnaB (GenBank accession number 078411); Ppu DnaB (GenBank accession number P51333); Sce VMA (GenBank accession number PXBYVA); Mf1 RecA (GenBank accession number not given); Mxe GyrA (GenBank accession number P72065); Ssp DnaE (GenBank accession number S76958 & S75328); and MIe DnaB (GenBank accession number CAA17948.1)

In other embodiments, inteins with alternative splicing mechanisms are preferred (see Southworth, et al., (2000) EMBO J., 19:5019-26). The GenBank accession numbers for inteins with alternative splicing mechanisms include, but are not limited to: Mja KIbA (GenBank accession number Q58191); and, Pfu KIbA (PF _{—}949263 in UMBI).

In yet other embodiments, inteins from thermophilic organisms are used. Random mutagenesis or directed evolution (i.e. PCR shuffling, etc.) of inteins from these organisms could lead to the isolation of temperature sensitive mutants. Thus, inteins from thermophiles (i.e., Archaea) which find use in the invention are: Mth RIR1 (GenBank accession number G69186); Pfu RIR1-1 (AAB36947.1); Psp-GBD Pol (GenBank accession number AAA67132.1); Thy Pol-2 (GenBank accession number CAC18555.1); Pfu IF2 (PF_{—}1088001 in UMBI); Pho Lon Baa29538.1); Mja r-Gyr (GenBank accession number G64488); Pho RFC (GenBank accession number F71231); Pab RFC-2 (GenBank accession number C75198); Mja RtcB (also referred to as Mja Hyp-2; GenBank accession number Q58095); and, Pho VMA (NT01 PH1971 in Tigr).

In addition to the ligation reactions outlined above, there are additional cross-linking reactions that allow for the fusion of the NAM enzyme and the candidate protein. For example, transglutaminases catalyze protein-to-protein cross-linking reactions (Lorand. (1996) Proc. Natl. Acad. Sci. USA, 93:24310-14313). The geometry of the cross-linked protein products depend that results from the cross-linking reaction depends on the number and spatial distribution of transglutaminase reactive glutamine and lysine residues in the protein substrates. Proteins with transglutaminase reactive glutamines are referred to as acceptor protein substrates, while proteins with lysine residues are referred to as donor protein substrates.

To participate in a transglutaminase-catalyzed reaction, glutamine residues must be part of a peptide or polypeptide (Kahlem, P., et al., (1996) Proc. Natl. Acad. Sci. USA, 93:14580-14585). It has long been known that in certain small proteins, most or all scattered gluatmine residues may act as amine acceptors, at least in the absence of secondary or tertiary structure preventing access of the enzyme. However, in native proteins, the nature of the neighboring residues has appreciable influence on the reactivity of a glutamine residue, with some residues being preferred to others. Among preferred glutamine residues are ones adjacent to as second glutamine residue.

In a preferred embodiment, a NAM enzyme-candidate protein fusion is made using a transglutaminase catalyzed cross-linking reaction. In this embodiment, polyglutamine residues may be added to the N- or C- terminus of either the NAM enzyme or the candidate protein to create an acceptor protein substrate. Between I and 6 glutamine residues may be added, with 2 residues being particularly preferred (Kahlem et al., supra). Donor protein substrates can be created by adding a lysine residue to the N- or C- terminus of either the NAM enzyme or the candidate protein.

In a preferred embodiment, an acceptor donor substrate comprising a NAM enzyme with polyglutamine residues is combined with a donor substrate comprising a candidate protein with a lysine residue. Cross-linking of the NAM enzyme to the candidate protein to form a fusion polypeptide is done under conditions that favor transglutaminase cross-linking (Kahlem et al., supra). As will be appreciated by those of skill in the art, the cross-linking reaction may be carried out in vitro by adding purified transglutaminase or in vivo.

It can be advantageous to construct the expression vector to provide further options to control attachment of the fusion enzyme to the EAS. For example, the EAS can be introduced into the nucleic acid molecule as two non-functional halves that are brought together following enzyme-mediated or non-enzyme-mediated homologous recombination, such as that mediated by cre-lox recombination, to form a functional EAS. Likewise, the referenced cre-lox consideration could also be used to control the formation of a functional fusion enzyme. The control of cre-lox recombination is preferably mediated by introducing the recombinase gene under the control of an inducible promoter into the expression system, whether on the same nucleic acid molecule or on another expression vector.

In a preferred embodiment, the expression vectors can also include components to ease in the enrichment and identification process of “hits” identified using the methods of the invention, as is more fully described below. In some embodiments, the covalent linkage between the NAM enzyme and the EAS sequence of the vector hinders the enrichment process (generally done through PCR) after a candidate protein has been identified as a hit. Accordingly, this embodiment relies on the use of recombinases and recombinase sites such as the cre/lox system and the FLP system (see for example the Creator™ Gene Cloning and Expression System sold by Clontech and the Gateway™ cloning system from Life Technologies). In this embodiment, the recombinase sites (e.g. the lox sites) are inserted downstream of the fusions (either prior to the creation of the fusions or afterwards). Panning and/or assays are run, as generally described below, to identify “hits”. These positive clone pools are purified (for example through phenol extraction and ethanol precipitation) and mixed with fresh vectors in the presence of the corresponding recombinase (for example the cre recombinase when lox sites are used). These recombinase reactions are very efficient and allow the “switching” of the candidate protein coding region from a NAP conjugate into a vector without a covalently attached NAM enzyme and candidate protein fusion. These plasmids can then be directly used for transformation of host cells without purification.

In addition to the NAM enzymes, candidate proteins, and linkers, the fusion nucleic acids can comprise additional coding sequences for other functionalities. As will be appreciated by those in the art, the discussion herein is directed to fusions of these other components to the fusion nucleic acids described herein; however, they can also be separate from the fusion protein and rather be a component of the expression vector comprising the fusion nucleic acid, as is generally outlined below.

Thus, in a preferred embodiment, the fusions are linked to a fusion partner. By “fusion partner” or “functional group” herein is meant a sequence that is associated with the candidate protein, that confers upon all members of the library in that class a common function or ability. Fusion partners can be heterologous (i.e. not native to the host cell), or synthetic (not native to any cell). Suitable fusion partners include, but are not limited to: a) presentation structures, as defined below, which provide the candidate proteins in a conformationally restricted or stable form, including hetero- or homodimerization or multimerization sequences; b) targeting sequences, defined below, which allow the localization of the candidate proteins into a subcellular or extracellular compartment or be incorporated into infected organisms, such as those infected by viruses or pathogens; c) rescue sequences as defined below, which allow the purification or isolation of the NAP conjugates; d) stability sequences, which confer stability or protection from degradation to the candidate protein or the nucleic acid encoding it, for example resistance to proteolytic degradation; e) linker sequences; or f) any combination of a), b), c), d), and e), as well as linker sequences as needed.

In a preferred embodiment, the fusion partner is a presentation structure. By “presentation structure” or grammatical equivalents herein is meant an amino acid sequence, which, when fused to candidate proteins, causes the candidate proteins to assume a conformationally restricted form. This is particularly useful when the candidate proteins are random, biased random or pseudorandom peptides. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems.

Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the randomized expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, dimerization sequences, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the randomized peptide on an exterior loop. See, for example, Myszka et al., Biochem. 33:2362-2373 (1994), hereby incorporated by reference, and FIG. 3). Using this system investigators have isolated peptides capable of high affinity interaction with the appropriate target. In general, coiled-coil structures allow for between 6 to 20 randomized positions. A preferred coiled-coil presentation structure is described in, for example, Martin et al., EMBO J. 13(22):5303-5309 (1994), incorporated by reference.

In a preferred embodiment, the presentation structure is a minibody structure. A “minibody” is essentially composed of a minimal antibody complementarity region. The minibody presentation structure generally provides two randomizing regions that in the folded protein are presented along a single face of the tertiary structure. See, for example, Bianchi et al., J. Mol. Biol. 236(2):649-59 (1994), and references cited therein, all of which are incorporated by reference. Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

A preferred minibody presentation structure is as follows: MGRNSQATSGFTFSHFYMEWVRGGEYIAASRHKHNKYTTEYSASVKGRYIVSRDTSQSILYLQKKKG PP (SEQ ID NO:1). The bold, underlined regions are the regions which may be randomized. The italized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXl sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly crosslinked structured conformations, similar to a presentation structure. Similarly, the randomization regions may be controlled to contain a certain number of residues to confer β-sheet or a-helical structures.

In one embodiment, the presentation structure is a dimerization or multimerization sequence. A dimerization sequence allows the non-covalent association of one candidate protein to another candidate protein, including peptides, with sufficient affinity to remain associated under normal physiological conditions. This effectively allows small libraries of candidate protein (for example, 10⁴) to become large libraries if two proteins per cell are generated which then dimerize, to form an effective library of 10⁸ (10⁴×10⁴). It also allows the formation of longer proteins, if needed, or more structurally complex molecules. The dimers may be homo- or heterodimers.

Dimerization sequences may be a single sequence that self-aggregates, or two sequences. That is, nucleic acids encoding both a first candidate protein with dimerization sequence 1, and a second candidate protein with dimerization sequence 2, such that upon introduction into a cell and expression of the nucleic acid, dimerization sequence 1 associates with dimerization sequence 2 to form a new structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

In a preferred embodiment, the fusion partner is a targeting sequence. As will be appreciated by those in the art, the localization of proteins within a cell is a simple method for increasing effective concentration and determining function. For example, RAF1 when localized to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane or within pathogens or viruses that have infected the cell; and b) extracellular locations via a secretory signal. Particularly preferred is localization to either subcellular locations or to the outside of the cell via secretion.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal; NFkB p50 (see, for example, Ghosh et al., Cell 62:1019 (1990)); NFkB p65 (see, for example, Nolan et al., Cell 64:961 (1991)); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (see, for example, Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462,1990.

In a preferred embodiment, the targeting sequence is a membrane anchoring signal sequence. This is particularly useful since many parasites and pathogens bind to the membrane, in addition to the fact that many intracellular events originate at the plasma membrane. Thus, membrane-bound peptide libraries are useful for both the identification of important elements in these processes as well as for the discovery of effective inhibitors. In addition, many drugs interact with membrane associated proteins. The invention provides methods for presenting the candidate proteins extracellularly or in the cytoplasmic space. For extracellular presentation, a membrane anchoring region is provided at the carboxyl terminus of the candidate protein. The candidate protein region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular candidate protein region is bound, confers a function on the cells (activation of a kinase, phosphatase, binding of other cellular components to effect function). Similarly, the candidate protein-containing region could be contained within a cytoplasmic region, and the transmembrane region and extracellular region remain constant or have a defined function.

In addition, it should be noted that in this embodiment, as well as others outlined herein, it is possible that the formation of the NAP conjugate happens after the screening; that is, having the fusion protein expressed on the extracellular surface means that it may not be available for binding to the nucleic acid. However, this may be done later, with lysis of the cell.

Membrane-anchoring sequences are well known in the art and are based on the genetic geometry of mammalian transmembrane molecules. Peptides are inserted into the membrane based on a signal sequence (designated herein as ssTM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

Membrane-anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful membrane-anchoring sequences include, for example, sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the transmembrane domain and 960-1382 are the cytoplasmic domain; see Hatakeyama, supra, and Ebina et al., Cell 40:747 (1985)); 2) class 11 integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (see, for example, Nakauchi et al., PNAS USA 82:5126 (1985) and 1-21 in the case of ICAM-2 (see, for example, Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (Nakauchi, supra) and 224-256 from ICAM-2 (Staunton, supra).

Alternatively, membrane anchoring sequences can include the GPI anchor, which results in a covalent bond between the molecule and the lipid bilayer via a glycosyl-phosphatidylinositol bond for example in DAF (see, for example, Homans et al., Nature 333(6170):269-72 (1988), and Moran et al., J. Biol. Chem. 266:1250 (1991)). In order to do this, the GPI sequence from Thy-1 can be inserted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function (see Cross et al., Mol. Cell. Biol. 4(9):1834 (1984); Spencer et al., Science 262:1019-1024 (1993), both of which are hereby incorporated by reference). This motif has already been shown to be effective in the localization of reporter genes and can be used to anchor the zeta chain of the TCR. This motif is placed 5′ of the variable region in order to localize the construct to the plasma membrane. Other modifications such as palmitoylation can be used to anchor constructs in the plasma membrane; for example, palmitoylation sequences from the G protein-coupled receptor kinase GRK6 sequence (see, for example, Stoffel et al., J. Biol. Chem 269:27791 (1994)); from rhodopsin (see, for example, Barnstable et al., J. Mol. Neurosci. 5(3):207 (1994)); and the p21 H-ras 1 protein (see, for example, Capon et al., Nature 302:33 (1983)).

In a preferred embodiment, the targeting sequence is a lysozomal targeting sequence, including, for example, a lysosomal degradation sequence such as Lamp-2 (KFERQ; Dice, Ann. N.Y. Acad. Sci. 674:58 (1992); or lysosomal membrane sequences from Lamp-1 (see, for example, Uthayakumar et al., Cell. Mol. Biol. Res. 41:405 (1995)) or Lamp-2 (see, for example, Konecki et la., Biochem. Biophys. Res. Comm. 205:1-5 (1994)).

Alternatively, the targeting sequence can comprise a mitrochondrial localization sequence, including mitochondrial matrix sequences (e.g. yeast alcohol dehydrogenase III; Schatz, Eur. J. Biochem. 165:1-6 (1987)); mitochondrial inner membrane sequences (yeast cytochrome c oxidase subunit IV; Schatz, supra); mitochondrial intermembrane space sequences (yeast cytochrome c1; Schatz, supra) or mitochondrial outer membrane sequences (yeast 70 kD outer membrane protein; Schatz, supra).

The target sequences also can comprise endoplasmic reticulum sequences, including the sequences from calreticulin (Pelham, Royal Society London Transactions B; 1-10 (1992)) or adenovirus E3/19K protein (see, for example, Jackson et al., EMBO J. 9:3153 (1990)).

Furthermore, targeting sequences also can include peroxisome sequences (for example, the peroxisome matrix sequence from Luciferase; Keller et al., PNAS USA 4:3264 (1987)); farnesylation sequences (for example, P21 H-ras 1; Capon, supra); geranylgeranylation sequences (for example, protein rab-5A; Farnsworth, PNAS USA 91:11963 (1994)); or destruction sequences (cyclin B1; Klotzbucher et al., EMBO J. 1:3053 (1996)).

In a preferred embodiment, the targeting sequence is a secretory signal sequence capable of effecting the secretion of the candidate protein. There are a large number of known secretory signal sequences which are placed 5′ to the variable peptide region, and are cleaved from the peptide region to effect secretion into the extracellular space. Secretory signal sequences and their transferability to unrelated proteins are well known, e.g., Silhavy, et al. (1985) Microbiol. Rev. 49, 398-418. This is particularly useful to generate a peptide capable of binding to the surface of, or affecting the physiology of, a target cell that is other than the host cell. In this manner, target cells grown in the vicinity of cells caused to express the library of peptides, are bathed in secreted peptide. Target cells exhibiting a physiological change in response to the presence of a peptide, e.g., by the peptide binding to a surface receptor or by being internalized and binding to intracellular targets, and the secreting cells are localized by any of a variety of selection schemes and the peptide causing the effect determined. Exemplary effects include variously that of a designer cytokine (i.e., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Similar to the membrane-anchored embodiment, it is possible that the formation of the NAP conjugate happens after the screening; that is, having the fusion protein secreted means that it may not be available for binding to the nucleic acid. However, this may be done later, with lysis of the cell. Suitable secretory sequences are known, including, for example, signals from IL-2 (see, for example, Villinger et al., J. Immunol. 155:3946 (1995)), growth hormone (see, for example, Roskam et al., Nucleic Acids Res. 7:30 (1979)); preproinsulin (see, for example, Bell et al., Nature 284:26 (1980)); and influenza HA protein (see, for example, Sekiwawa et al., PNAS 80:3563)). A particularly preferred secretory signal sequence is the signal leader sequence from the secreted cytokine IL-4.

In a preferred embodiment, the fusion partner is a rescue sequence (sometimes also referred to herein as “purification tags” or “retrieval properties”). A rescue sequence is a sequence which may be used to purify or isolate either the candidate protein or the NAP conjugate. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST. Rescue sequences can be utilized on the basis of a binding event, an enzymatic event, a physical property or a chemical property.

Alternatively, the rescue sequence can comprise a unique oligonucleotide sequence which serves as a probe target site to allow the quick and easy isolation of the construct, via PCR, related techniques, or hybridization.

In a preferred embodiment, the fusion partner is a stability sequence to confer stability to the candidate protein or the nucleic acid encoding it. Thus, for example, peptides can be stabilized by the incorporation of glycines after the initiation methionine, for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate protein structure. Thus, preferred stability sequences are as follows: MG(X)nGGPP, where X is any amino acid and n is an integer of at least four.

In addition, linker sequences, as defined above, may be used in any configuration as needed.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

Combinations of fusion partners can be used if desired. Thus, for example, any number of combinations of presentation structures, targeting sequences, rescue sequences, and stability sequences may be used, with or without linker sequences. Similarly, as discussed herein, the fusion partners may be associated with any component of the expression vectors described herein: they may be directly fused with either the NAM enzyme, the candidate protein, or the EAS, described below, or be separate from these components and contained within the expression vector.

In addition to sequences encoding NAM enzymes and candidate proteins, and the optional fusion partners, the nucleic acids of the invention preferably comprise an enzyme attachment sequence. By “enzyme attachment sequence” or “EAS” herein is meant selected nucleic acid sequences that mediate attachment with NAM enzymes. Such EAS nucleic acid sequences possess the specific sequence or specific chemical or structural configuration that allows for attachment of the NAM enzyme and the EAS. The EAS can comprise DNA or RNA sequences in their natural conformation, or hybrids. EASs also can comprise modified nucleic acid sequences or synthetic sequences inserted into the nucleic acid molecule of the present invention. EASs also can comprise non-natural bases or hybrid non-natural and natural (i.e., found in nature) bases.

As will be appreciated by those in the art, the choice of the EAS will depend on the NAM enzyme, as individual NAM enzymes recognize specific sequences and thus their use is paired. Thus, suitable NAM/EAS pairs are the sequences recognized by Rep proteins (sometimes referred to herein as “Rep EASs”) and the Rep proteins, the H-1 recognition sequence and H-1, etc. In addition, EASs can be utilized which mediate improved covalent binding with the NAM enzyme compared to the wild-type or naturally occurring EAS.

In a preferred embodiment, the EAS is double-stranded. By way of example, a suitable EAS is a double-stranded nucleic acid sequence containing specific features for interacting with corresponding NAM enzymes. For example, Rep68 and Rep78 recognize an EAS contained within an AAV ITR, the sequence of which is set forth in Example 1. In addition, these Rep proteins have been shown to recognize an ITR-like region in human chromosome 19 as well, the sequence of which is shown in FIG. 48.

An EAS also can comprise supercoiled DNA with which a topoisomerase interacts and forms covalent intermediate complexes. Alternatively, an EAS is a restriction enzyme site recognized by an altered restriction enzyme capable of forming covalent linkages. Finally, an EAS can comprise an RNA sequence and/or structure with which specific proteins interact and form stable complexes (see, for example, Romaniuk and Uhlenbeck, Biochemistry, 24, 4239-44 (1985)).

In a preferred embodiment, the EAS is an RNA sequence and RNA-protein fusions are made.

Preferably, RNA-protein fusions are made by fusing a gene encoding a NAM enzyme (described above) to either the N- or C-terminal of a gene encoding a candidate protein to create a fusion nucleic acid. An EAS specific for the NAM enzyme may be inserted in either the 5′ UTR and/or the 3′ UTR of the fusion nucleic acid. As shown in FIG. 50, as the fusion nucleic acid is translated, the newly translated NAM protein covalently binds to the EAS, thereby creating an RNA-protein fusion.

The present invention relies on the specific binding of the NAM enzyme to the EAS in order to mediate linkage of the fusion enzyme to the nucleic acid molecule. One of ordinary skill in the art will appreciate that use of an EAS consisting of a small nucleic acid sequence would result in non-specific binding of the NAM enzyme to expression vectors and the host cell genome depending on the frequency that the accessible EAS motif appears in the vector or host genome. Therefore, the EAS of the present invention is preferably comprised of a nucleic acid sequence of sufficient length such that specific fusion protein-coding nucleic acid molecule attachment results. For example, the EAS is preferably greater than five nucleotides in length. More preferably, the EAS is greater than 10 nucleotides in length, e.g., with EASs of at least 12, 15, 20, 25, 30, 35, 40, 45 or 50 nucleotides being preferred.

Moreover, preferably the EAS is present in the host cell genome in a very limited manner, such that at most, only one or two NAM enzymes can bind per genome, e.g. no more than once in a human cell genome. In situations wherein the EAS is present many times within a host cell, e.g., a human cell genome, the probability of fusion proteins encoded by the expression vector attaching to the host cell genome and not the expression vector increases and is therefore undesirable. For instance, the bacteriophage P2 A protein recognizes a relatively short DNA recognition sequence. As such, use of the P2 A protein in mammalian cells would result in protein binding throughout the host genome, and identification of the desired nucleic acid sequence would be difficult. Thus, preferred embodiments exclude the use of P2A as a NAM enzyme.

One of ordinary skill in the art will appreciate that the NAM enzyme used in the present invention or the corresponding EAS can be manipulated in order to increase the stability of the fusion protein-nucleic acid molecule complex. Such manipulations are contemplated herein, so long as the NAM enzyme forms a covalent bond with its corresponding EAS.

In a preferred embodiment, the nucleic acids of the invention preferably comprise a DNA binding motif. By “DNA binding motif” herein is meant selected nucleic acid sequences that mediate attachment of small molecule conjugates. The DNA binding motif should posses a sequence, or a specific chemical or structural configuration to allow for the attachment of a small molecule conjugate. The DNA binding motif may comprise DNA sequences in their natural conformation or hybrids. The DNA binding motif also can comprise modified nucleic acid sequences or synthetic sequences, non-natural bases or hybrid non-natural and natural bases.

Suitable DNA binding motifs include, but are not limited to, binding sequences capable of binding small molecule conjugates; for example, molecules that can be combined in antiparallel, side-by-side, dimeric complexes or in hairpin or cyclic configurations. Preferably, DNA binding motifs are between 4 to 20 base pairs. Accordingly, the DNA binding motifs of the present invention may be one of any of the following lengths: 4 base pairs, 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, and 20 base pairs in length. Binding motifs of 5 to 7 base pairs are advantageous as binding affinity for small molecule conjugates, especially polyamides, is high. See Dervan and Bürli, (1999) Curr. Opin. Chem. Biol. 3:688-693, hereby incorporated by reference in its entirety.

In a preferred embodiment, the DNA sequence of the binding motif comprises (A/T)G(A/T)C(A/T). Other suitable DNA sequences include, but are not limited to, (A/T)G(A/T)₃; GTACA; TGTACA; TGTGTA; TGTAACA; TGTTATTGTTA; and other suitable sequences described in Dervan and Bürli, supra; Mapp, et al., (2000) Proc. Natl. Acad. Sci. USA, 97:3930-3935.

By “small molecule conjugate” herein is meant a small molecule that comprises at least two domains. The first domain comprises a moiety capable of recognizing DNA in a sequence specific manner, referred to herein as a “DNA binding moiety”. By “DNA binding moiety” herein is synthetic ligand that recognizes and binds too DNA. That is, the ligand is capable of recognizing and binding to specific sequences in either the major or minor groove of DNA (Dervan and Bürli, supra)..

In a preferred embodiment, the synthetic ligand will recognize and bind to the minor groove of DNA. Suitable ligands for binding to the minor groove of DNA include, but are not limited to polyamides. Suitable polyamides include, but are not limited to, synthetic peptides containing non-natural amino acids, N-tmethyl-imidazole, N-methyl-pyrrole, N-methyl-3-hydroxypyrrole (Hp), and the amino acid beta-alanine. Synthetic ligands are preferably designed using the pairing rules for polyamide binding to DNA (Dervan and Bürli, supra.) Thus, in an anti-parallel, side-by-side motif, a pyrrole (Py) opposite an imidazole (Im; Py/Im pairing) targets a C-G base pair (bp), whereas an Im/Py pair recognizes a G-C bp/ A Py/Py pair is degenerated and binds both A-T and T-A pairs in preference to G-C/C-G pairs. The A-T/T-A degeneracy by Py/Py can be avoided by using an Hp/Py pair. An Hp/Py pair recognizes a T-A bp whereas a Py/Hp pair targets an A-T bp.

Synthetic ligands comprising polyamides may be synthesized as cyclic or hairpin structures, tandem hairpins, H-pins, or as unlinked dimers (homo or heterodimers). Hairpin structures are preferred, as they provide high affinity and specificity, especially as the number of heterocyclic units are increased. Hairpin structures may be created by connecting the carboxyl and amino terminal of two adjacent polyamides with a γ-butyric acid linker (see disclosure 2 paragraphs below and conform e.g. chiral). A carboxy-terminal β-linker element, such as a β-alanine reside may be used to specify for A-T in preference to G-C (Dervan and Bürli, supra) with increased DNA affinity. For example, hairpin structures of core sequence composition ImPyPy-γ-PyPyPy may be used coding to G A/T A/T A/T.

Other useful hairpin structures have core sequence compositions comprising eight Im and Py rings linked with a y-butyric acid linker and terminate in a β-alanine residue. In addition, hairpin structures may be created using Hp-Im-Py motifs. In addition, cooperatively binding hairpin polyamide ligands, which bind in a homo or hetero dimeric fashion can be designed (see Dervan and Bürli, supra).

In a preferred embodiment, synthetic ligands containing Im and Py are combined in anti-parallel, unlinked side-by-side dimeric complexes, which may consist of homo or hetero dimers, for the recognition of longer sequences. A β-alanine residue can be used to join adjacent polyamide subunits to provide fully overlapping or partially overlapping extended homodimers recognizing between 10 to 20 bp (see Dervan and Bürli, supra).

In a preferred embodiment, chiral turn, cyclic or β/ring pair polyamide synthetic ligands can be designed. These ligands are especially used for binding to DNA sequences that exhibit microstructure (see Dervan and Bürli, supra).

The second domain comprises a “rescue tag” as defined below. The two domains may be contiguous or separated by linker sequence as defined below. In addition, rescue sequences can rely on the use of triplex helix for motion, with high stabilities, using naturally occurring nucleosides of analogs such as PNA.

In addition, as outlined below, the fusion nucleic acids can also comprise capture sequences that hybridize to capture probes on a surface, to allow the formation of support bound NAP conjugates and specifically arrays of the conjugates.

In addition to the components outlined herein, including NAM enzyme-candidate protein fusions, EASs, linkers, fusion partners, etc., the expression vectors may comprise a number of additional components, including, selection genes as outlined herein (particularly including growth-promoting or growth-inhibiting functions), activatible elements, recombination signals (e.g. cre and lox sites) and labels.

Preferably, the present invention fusion peptide, fusion nucleic acid, conjugates, etc., further comprise a labeling component. Again, as for the fusion partners of the invention, the label can be fused to one or more of the other components, for example to the NAM fusion protein, in the case where the NAM enzyme and the candidate protein remain attached, or to either component, in the case where scission occurs, or separately, under its own promoter. In addition, as is further described below, other components of the assay systems may be labeled.

Labels can be either direct or indirect detection labels, sometimes referred to herein as “primary” and “secondary” labels. By “detection label” or “detectable label” herein is meant a moiety that allows detection. This may be a primary label or a secondary label. Accordingly, detection labels may be primary labels (i.e. directly detectable) or secondary labels (indirectly detectable).

In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles. In a preferred embodiment, the detection label is a primary label. A primary label is one that can be directly detected, such as a fluorophore.

Preferred labels include, for example, chromophores or phosphors but are preferably fluorescent dyes or moieties. Fluorophores can be either “small molecule” fluors, or proteinaceous fluors. In a preferred embodiment, particularly for labeling of target molecules, as described below, suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference.

In a preferred embodiment, for example when the label is attached to the fusion polypeptide or is to be expressed as a component of the expression vector, proteinaceous fluores are used. Suitable autofluorescent proteins include, but are not limited to, the green fluorescent protein (GFP) from Aequorea and variants thereof; including, but not limited to, GFP, (Chalfie, et al., Science 263(5148):802-805 (1994)); enhanced GFP (EGFP; Clontech-Genbank Accession Number U55762 )), blue fluorescent protein (BFP; Quantum Biotechnologies, Inc. 1801 de Maisonneuve Blvd. West, 8th Floor, Montreal (Quebec) Canada H3H 1J9; Stauber, R. H. Biotechniques 24(3):462-471 (1998);

Heim, R. and Tsien, R. Y. Curr. Biol. 6:178-182 (1996)), and enhanced yellow fluorescent protein (EYFP; Clontech Laboratories, Inc., 1020 East Meadow Circle, Palo Alto, Calif. 94303). In addition, there are recent reports of autofluorescent proteins from Renilla and Ptilosarcus species. See WO 30 92/15673; WO 95/07463; WO 98/14605; WO 98/26277; WO 99/49019; U.S. Pat. No. 5,292,658; U.S Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S Pat. No. 5,876,995; and U.S. Pat. No. 5,925,558; all of which are expressly incorporated herein by reference.

In a preferred embodiment, the label protein is Aequorea green fluorescent protein or one of its variants; see Cody et al., Biochemistry 32:1212-1218 (1993); and Inouye and Tsuji, FEBS Lett. 341:277-280 (1994), both of which are expressly incorporated by reference herein.

In a preferred embodiment, a secondary detectable label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; enzymes such as horseradish peroxidase, alkaline phosphatases, luciferases, etc; and cell surface markers, etc.

In a preferred embodiment, the secondary label is a binding partner pair. For example, the label may be a hapten or antigen, which will bind its binding partner. In a preferred embodiment, the binding partner can be attached to a solid support to allow separation of components containing the label and those that do not. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. In general, the smaller of the pair is attached to the system component for incorporation into the assay, although this is not required in all embodiments. Preferred binding partner pairs include, but are not limited to, biotin (or imino-biotin) and streptavidin, digeoxinin and Abs, etc.

In a preferred embodiment, the binding partner pair comprises a primary detection label (for example, attached to the assay component) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶ M⁻¹, with less than about 10⁻⁵-10⁻⁹ M⁻¹, being preferred and less than about 10⁻⁷-10⁻⁹ M⁻¹ being particularly preferred.

In a preferred embodiment, the secondary label is a chemically modifiable moiety. In this embodiment, labels comprising reactive functional groups are incorporated into the assay component. The functional group can then be subsequently labeled with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference).

Thus, in a preferred embodiment, the nucleic acids of the invention comprise (i) a fusion nucleic acid comprising sequences encoding a NAM enzyme and a candidate protein, and (ii) an EAS. These nucleic acids are preferably incorporated into an expression vector; thus providing libraries of expression vectors, sometimes referred to herein as “NAM enzyme expression vectors”.

The expression vectors may be either self-replicating extrachromosomal vectors, vectors which integrate into a host genome, or linear nucleic acids that may or may not self-replicate. Thus, specifically included within the definition of expression vectors are linear nucleic acid molecules. Expression vectors thus include plasmids, plasmid-liposome complexes, phage vectors, and viral vectors, e.g., adeno-associated virus (AAV)-based vectors, retroviral vectors, herpes simplex virus (HSV)-based vectors, and adenovirus-based vectors. The nucleic acid molecule and any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994) Generally, these expression vectors include transcriptional and translational regulatory nucleic acid sequences operably linked to the nucleic acid encoding the NAM protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the NAM protein, as will be appreciated by those in the art; for example, transcriptional and translational regulatory nucleic acid sequences from Bacillus are preferably used to express the NAM protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer, silencer, or activator sequences. In a preferred embodiment, the regulatory sequences include a promoter and transcriptional start and stop sequences.

A “promoter” is a nucleic acid sequence that directs the binding of RNA polymerase and thereby promotes RNA synthesis. Promoter sequences include constitutive and inducible promoter sequences. Exemplary constitutive promoters include, but are not limited to, the CMV immediate-early promoter, the RSV long terminal repeat, mouse mammary tumor virus (MMTV) promoters, etc. Suitable inducible promoters include, but are not limited to, the IL-8 promoter, the metallothionine inducible promoter system, the bacterial lacZYA expression system, the tetracycline expression system, and the T7 polymerases system. The promoters can be either naturally occurring promoters, hybrid promoters, or synthetic promoters. Hybrid promoters, which combine elements of more than one promoter, are also known in the art, and are useful in the present invention.

In addition, the expression vector may comprise additional elements. For example, the expression vector may have two replication systems (e.g., origins of replication), thus allowing it to be maintained in two organisms, for example in animal cells for expression and in a prokaryotic host for cloning and amplification. Furthermore, for integrating expression vectors, which are generally not preferred in most embodiments, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7(Clifton: Humana Press, 1991).

It should be noted that the compositions and methods of the present invention allow for specific chromosomal isolation. For example, since human chromosome 19 contains a Rep-binding sequence (e.g. an EAS), a NAP conjugate will be formed with chromosome 19, when the NAM enzyme is Rep. Cell lysis followed by immunoprecipitation, either using antibodies to the Rep protein itself (e.g. no candidate protein is necessary) or to a fused candidate protein or purification tag, allows the purification of the chromosome. This is a significant advance over current chromosome purification techniques. Thus, by selectively or non-selectively integrating EAS sites into chromosomes, different chromosomes may be purified.

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers new phenotypes of the cells which contain the vector. These phenotypes include, for instance, enhanced or decreased cell growth. The phenotypes can also include resistance to a selection agent. Suitable selection agents include, but are not limited to, neomycin (or its analog G418), blasticidin S, histinidol D, bleomycin, puromycin, hygromycin B, and other drugs. The expression vector also can comprise a coding sequence for a marker protein, such as the green fluorescence protein, which enables, for example, rapid identification of successfully transduced cells.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

One expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al.,Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

The fusion proteins of the present invention can be produced by culturing a host cell transformed with nucleic acid, preferably an expression vector as outlined herein, under the appropriate conditions to induce or cause production of the fusion protein. The conditions appropriate for fusion protein production will vary with the choice of the expression vector and the host cell, and will be easily ascertained by one skilled in the art using routine methods. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cells are lytic viruses, and thus harvest time selection can be crucial for product yield.

Any host cell capable of withstanding introduction of exogenous DNA and subsequent protein production is suitable for the present invention. The choice of the host cell will depend, in part, on the assay to be run; e.g., in vitro systems may allow the use of any number of procaryotic or eucaryotic organisms, while ex vivo systems preferably utilize animal cells, particularly mammalian cells with a special emphasis on human cells. Thus, appropriate host cells include yeast, bacteria, archaebacteria, plant, and insect and animal cells, including mammalian cells and particularly human cells. The host cells may be native cells, primary cells, including those isolated from diseased tissues or organisms, cell lines (again those originating with diseased tissues), genetically altered cells, etc. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, etc. See the ATCC cell line catalog, hereby expressly incorporated by reference.

In a preferred embodiment, the fusion proteins are expressed in mammalian cells. Mammalian expression systems are also known in the art, and include, for example, retroviral and adenoviral systems. A mammalian promoter is any DNA sequence capable of binding mammalian RNA polymerase and initiating the downstream (3′) transcription of a coding sequence for a fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived from SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei. In a preferred embodiment, protoplast fusion methods are used. This method involves the removal of the cell wall material, resulting in membrane exposed clels (known as protoplasts or spheroplasts). These are placed in contact with another cell resulting in fusion. See Sandri-Goldin et al., Methods in Enzymology 101:401, 1983 and Seed et al. PNAS 84:3365 (1987).

In a preferred embodiment, NAM fusions are produced in bacterial systems. Bacterial expression systems are widely available and include, for example, plasmids.

A suitable bacterial promoter is any nucleic acid sequence capable of binding bacterial RNA polymerase and initiating the downstream (3′) transcription of the coding sequence of the fusion into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the fusion proteins in bacteria or other cells. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

Suitable bacterial cells include, for example, vectors for Bacillus subtilis, E. coli, Streptococcus cremoris, and Streptococcus lividans, among others. The bacterial expression vectors can be transformed into bacterial host cells using techniques well known in the art, such as calcium chloride treatment, electroporation, and others. One benefit of using bacterial cells in the ability to propagate the cells comprising the expression vectors, thus generating clonal populations.

NAM fusion proteins also can be produced in insect cells such as Sf9 cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In addition, NAM fusion proteins can be produced in yeast cells. Yeast expression systems are well known in the art, and include, for example, expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions. One benefit of using yeast cells is the ability to propagate the cells comprising the vectors, thus generating clonal populations.

Preferred expression vectors are shown in FIGS. 49A-49N.

In general, once the expression vectors of the invention are made, they can follow one of two fates, which are merely exemplary: they are introduced into cell-free translation systems, to create libraries of nucleic acid/protein (NAP) conjugates that are assayed in vitro, or, preferably they are introduced into host cells where the NAP conjugates are formed; the cells may be optionally lysed and assayed accordingly.

In a preferred embodiment, the expression vectors are made and introduced into cell-free systems for translation, followed by the attachment of the NAP enzyme to the EAS, forming a nucleic acid/protein (NAP) conjugate. By “nucleic acid/protein conjugate” or “NAP conjugate” herein is meant a covalent attachment between the NAP enzyme and the EAS, such that the expression vector comprising the EAS is covalently attached to the NAP enzyme. Suitable cell free translation systems are known in the art. Once made, the NAP conjugates are used in assays as outlined below.

In a preferred embodiment, the expression vectors of the invention are introduced into host cells as outlined herein. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, gene guns, etc. The candidate nucleic acids may stably integrate into the genome of the host cell (for example, with retroviral introduction, outlined herein) or may exist either transiently or stably in the cytoplasm (i.e. through the use of traditional plasmids, utilizing standard regulatory sequences, selection markers, etc.). Suitable host cells are outlined above, with eucaryotic, mammalian and human cells all preferred.

Many previously described methods involve peptide library expression in bacterial cells. Yet, it is understood in the art that translational machinery such as codon preference, protein folding machinery, and post-translational modifications of, for example, mammalian peptides, are unachievable or altered in bacterial cells, if such modifications occur at all. Peptide library screening in bacterial cells often involves expression of short amino acid sequences, which can not imitate a protein in its natural configuration. Screening of these small, sub-part sequences cannot effectively determine the function of a native protein in that the requirements for, for instance, recognition of a small ligand for its receptor, are easily satisfied by small sequences without native conformation. The complexities of tertiary structure are not accounted for, thereby easing the requirements for binding.

One advantage of the present invention is the ability to express and screen unknown peptides in their native environment and in their native protein conformation. The covalent attachment of the fusion enzyme to its corresponding expression vector allows screening of peptides in organisms other than bacteria. Once introduced into a eukaryotic host cell, the nucleic acid molecule is transported into the nucleus where replication and transcription occurs. The transcription product is transferred to the cytoplasm for translation and post-translational modifications. However, the produced peptide and corresponding nucleic acid molecule must meet in order for attachment to occur, which is hindered by the compartmentalization of eukaryotic cells. NAM enzyme-EAS recognition can occur in four ways, which are merely exemplary and do not limit the present invention in any way. First, the host cells can be allowed to undergo one round of division, during which the nuclear envelope breaks down. Second, the host cells can be infected with viruses that perforate the nuclear envelope. Third, specific nuclear localization or transporting signals can be introduced into the fusion enzyme. Finally, host cell organelles can be disrupted using methods known in the art.

The end result of the above-described approaches is the transfer of the expression vector into the same environment as the fusion enzyme. The non-covalent interaction between a DNA binding protein and attachment site of previously described expression libraries would not survive the procedures required to allow linkage of the fusion protein to its expression vector in eukaryotic cells. Other DNA-protein linkages described in the art, such as those using the bacterial P2 A DNA binding peptide, require the binding peptide to remain in direct contact with its coding DNA in order for binding to occur, i.e., translation must occur proximal to the coding sequence (see, for example, Lindahl, Virology, 42, 522-533 (1970)). Such linkages are only achievable in prokaryotic systems and cannot be produced in eukaryotic cells.

Once the NAM enzyme expression vectors have been introduced into the host cells, the cells are optionally lysed. Cell lysis is accomplished by any suitable technique, such as any of a variety of techniques known in the art (see, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994), hereby expressly incorporated by reference). Most methods of cell lysis involve exposure to chemical, enzymatic, or mechanical stress. Although the attachment of the fusion enzyme to its coding nucleic acid molecule is a covalent linkage, and can therefore withstand more varied conditions than non-covalent bonds, care should be taken to ensure that the fusion enzyme-nucleic acid molecule complexes remain intact, i.e., the fusion enzyme remains associated with the expression vector.

In a preferred embodiment, the NAP conjugate may be purified or isolated after lysis of the cells. Ideally, the lysate containing the fusion protein-nucleic acid molecule complexes is separated from a majority of the resulting cellular debris in order to facilitate interaction with the target. For example, the NAP conjugate may be isolated or purified away from some or all of the proteins and compounds with which it is normally found after expression, and thus may be substantially pure. For example, an isolated NAP conjugate is unaccompanied by at least some of the material with which it is normally associated in its natural (unpurified) state, preferably constituting at least about 0.5%, more preferably at least about 5% by weight or more of the total protein in a given sample. A substantially pure protein comprises at least about 75% by weight or more of the total protein, with at least about 80% or more being preferred, and at least about 90% or more being particularly preferred.

NAP conjugates may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, gel filtration, and chromatofocusing. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the NAP conjugate. In some instances no purification will be necessary.

Thus, the invention provides for NAP conjugates that are either in solution, optionally purified or isolated, or contained within host cells. Once expressed and purified if necessary, the NAP conjugates are useful in a number of applications, including in vitro and ex vivo screening techniques. One of ordinary skill in the art will appreciate that both in vitro and ex vivo embodiments of the present inventive method have utility in a number of fields of study. For example, the present invention has utility in diagnostic assays and can be employed for research in numerous disciplines, including, but not limited to, clinical pharmacology, functional genomics, pharamcogenomics, agricultural chemicals, environmental safety assessment, chemical sensor, nutrient biology, cosmetic research, and enzymology.

In a preferred embodiment, the NAP conjugates are used in in vitro screening techniques. In this embodiment, the NAP conjugates are made and screened for binding and/or modulation of bioactivites of target molecules. One of the strengths of the present invention is to allow the identification of target molecules that bind to the candidate proteins. As is more fully outlined below, this has a wide variety of applications, including elucidating members of a signaling pathway, elucidating the binding partners of a drug or other compound of interest, etc.

Thus, the NAP conjugates are used in assays with target molecules. By “target molecules” or grammatical equivalents herein is meant a molecule for which an interaction is sought; this term will be generally understood by those in the art. Target molecules include both biological and non-biological targets. Biological targets refer to any defined and non-defined biological particles, such as macromolecular complexes, including viruses, cells, tissues and combinations, that are produced as a result of biological reactions in cells. Non-biological targets refer to molecules or structure that are made outside of cells as a result of either human or non-human activity. The inventive library can also be applied to both chemically defined targets and chemically non-defined targets. “Chemically defined targets” refer to those targets with known chemical nature and/or composition; “chemically non-defined targets” refer to targets that have either unknown or partially known chemical nature/composition.

Thus, suitable target molecules encompass a wide variety of different classes, including, but not limited to, cells, viruses, proteins (particularly including enzymes, cell-surface receptors, ion channels, and transcription factors, and proteins produced by disease-causing genes or expressed during disease states), carbohydrates, fatty acids and lipids, nucleic acids, chemical moieties such as small molecules, agricultural chemicals, drugs, ions (particularly metal ions), polymers and other biomaterials. Thus for example, binding to polymers (both naturally occurring and synthetic), or other biomaterials, may be done using the methods and compositions of the invention.

In one aspect, the target is a nucleic acid sequence and the desired candidate protein has the ability to bind to the nucleic acid sequence. The present invention is well suited for identification of DNA binding peptides and their coding sequences, as well as the target nucleic acids that are recognized and bound by the DNA binding peptides. It is known that DNA-protein interactions play important roles in controlling gene expression and chromosomal structure, thereby determining the overall genetic program in a given cell. It is estimated that only 5% of the human genome is involved in coding proteins. Thus, the remaining 95% may be sites with which DNA binding proteins interact, thereby controlling a variety of genetic programs such as regulation of gene expression. While the number of DNA binding peptides present in the human genome is not known, the complete sequence information now available for many genomes has revealed the full “substrate,” that is, the entire repertoire of DNA sequences with which DNA binding peptides may interact. Thus, it would be advantageous in genetic research to (1) identify nucleic acid sequences that encode DNA binding peptides, and (2) determine the substrate of these DNA binding peptides.

Current approaches used in determining protein-DNA interactions are focused on studying the individual interactions between DNA and specific protein targets. A variety of biochemical and molecular assays including DNA footprinting, nuclease protection, gel shift, and affinity chromatographic binding are employed to study protein-DNA interactions. Although these methods are useful for detecting individual DNA-protein interactions, they are not suitable for large-scale analyses of these interactions at the genomic level. Thus, there is a need in the art to perform large-scale analyses of DNA binding proteins and their interacting DNA sequences. The methods and libraries of the present invention are useful for such analyses. For example, the fusion enzyme library encoding potential DNA binding peptides can be screened against a population of target DNA segments. The population of target DNA segments can be, for instance, random DNA, fragmented genomic DNA, degenerate sequences, or DNA sequences of various primary, secondary or tertiary structures. The specificity of the DNA binding peptide-substrate binding can be varied by changing the length of the recognition sequence of the target DNA, if desired. Binding of the potential DNA binding peptide to a member of the population of target DNA segments is detected, and further study of the particular DNA recognition sequence bound by the DNA binding peptide can be performed. To facilitate identification of fusion enzyme-target nucleic acid complexes, the population of DNA segments can be bound to, for example, beads or constructed as DNA arrays on microchips. Therefore, using the present inventive method, one of ordinary skill in the art can identify DNA binding peptides, identify the coding sequence of the DNA binding peptides, and determine what nucleic acid sequence the DNA binding peptides recognize and bind. Thus, in one embodiment, the present invention provides methods for creating a map of DNA binding sequences and DNA binding proteins according to their relative positions, to provide chromosome maps annotated with proteins and sequences. A database comprising such information would then allow for correlating gene expression profiles, disease phenotype, pharmacogenomic data, and the like.

Thus, the NAP conjugates are used in screens to assay binding to target molecules and/or to screen candidate agents for the ability to modulate the activity of the target molecule.

In general, screens are designed to first find candidate proteins that can bind to target molecules, and then these proteins are used in assays that evaluate the ability of the candidate protein to modulate the target's bioactivity. Thus, there are a number of different assays which may be run; binding assays and activity assays. As will be appreciated by those in the art, these assays may be run in a variety of configurations, including both solution-based assays and utilizing support-based systems.

In a preferred embodiment, the assays comprise combining the NAP conjugates of the invention and a target molecule, and determining the binding of the candidate protein of the NAP conjugate to the target molecule. Preferably, libraries of NAP conjugates (e.g. comprising a library of different candidate proteins) is contacted with either a single type of target molecule, a plurality of target molecules, or one or more libraries of target molecules.

In a preferred embodiment, the detection of the interactions of candidate ligands with candidate proteins can be detected using non-denaturing gel electrophoresis. In this embodiment, the target ligand is linked to either a primary or secondary label as outlined herein. The labeled target ligand (or libraries of such ligands) is then incubated with a NAP conjugate library and run on a non-denaturing gel as is well known in the art. The visualization of the label allows the excision of the relevant bands followed by isolation of the NAP-conjugate using the techniques outlined herein such as PCR amplification), which can then be verified or used in additional rounds of panning.

Generally, in a preferred embodiment of the methods herein, one of the components of the invention, either the NAP conjugate or the target molecule, is non-diffusably bound to an insoluble support having isolated sample receiving areas (e.g. a microtiter plate, an array, etc.). The insoluble support may be made of any composition to which the assay component can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon®, etc. Microtiter plates and arrays are especially convenient because a large number of assays can be carried out simultaneously, using small amounts of reagents and samples. Alternatively, bead-based assays may be used, particularly with use with fluorescence activated cell sorting (FACS). The particular manner of binding the assay component is not crucial so long as it is compatible with the reagents and overall methods of the invention, maintains the activity of the composition and is nondiffusable.

In a preferred embodiment, the NAP conjugates of the invention are arrayed as is generally outlined in U.S. Ser. Nos. 09/792,405 and 09/792,630, filed Feb. 22, 2001, both of which are expressly incorporated by reference. In this embodiment, NAP vectors that also contain capture sequences that will hybridize with capture probes on the surface of a biochip are used, such that the NAP conjugates can be “captured” or “arrayed” on the biochip. These protein biochips can then be used in a wide variety of ways, including diagnosis (e.g. detecting the presence of specific target analytes), screening (looking for target analytes that bind to specific proteins), and single-nucleotide polymorphism (SNP) analysis.

Alternatively, the target analytes can be arrayed on a biochip and the NAP conjugates panned against these biochips.

As will be appreciated by those in the art, in these biochip formats, it is preferable that the soluble component of the assay be labeled. This can be done in a wide variety of ways, as will be appreciated by those in the art. For example, in the case where the target analytes or test ligands are arrayed, the NAP conjugates can contain a fusion partner comprising a primary or secondary label. Preferred embodiments utilize autofluorescent proteins, including, but not limited to, green fluorescent proteins and derivatives from Aqueorea species, Ptil.** species, and Renilla species. Alternatively, when the NAP conjugates are arrayed, generally through the use of capture sequences that will hybridize to capture probes on a surface, the target analytes can be labeled, again using any number of primary or secondary labels as defined herein.

Accordingly, the present invention provides biochips comprising a substrate with an array of molecules. By “biochip” or “array” herein is meant a substrate with a plurality of biomolecules in an array format; the size of the array will depend on the composition and end use of the array.

The biochips comprise a substrate. By “substrate” or “solid support” or other grammatical equivalents herein is meant any material appropriate for the attachment of capture probes and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, ceramics, and a variety of other polymers. In a preferred embodiment, the substrates allow optical detection and do not themselves appreciably fluoresce.

In addition, as is known the art, the substrate may be coated with any number of materials, including polymers, such as dextrans, acrylamides, gelatins, agarose, biocompatible substances such as proteins including bovine and other mammalian serum albumin, etc.

Preferred substrates include silicon, glass, polystyrene and other plastics and acrylics.

Generally the substrate is flat (planar), although as will be appreciated by those in the art, other configurations of substrates may be used as well, including the placement of the probes on the inside surface of a tube, for flow-through sample analysis to minimize sample volume.

The present system finds particular utility in array formats, i.e. wherein there is a matrix of addressable locations (herein generally referred to “pads”, “addresses” or “micro-locations”). By “array” herein is meant a plurality of capture probes in an array format; the size of the array will depend on the composition and end use of the array. Arrays containing from about 2 different capture probes to many thousands can be made. Generally, the array will comprise from two to as many as 100,000 or more, depending on the size of the pads, as well as the end use of the array. Preferred ranges are from about 2 to about 10,000, with from about 5 to about 1000 being preferred, and from about 10 to about 100 being particularly preferred. In some embodiments, the compositions of the invention may not be in array format; that is, for some embodiments, compositions comprising a single capture probe may be made as well. In addition, in some arrays, multiple substrates may be used, either of different or identical compositions. Thus for example, large arrays may comprise a plurality of smaller substrates.

In one embodiment, e.g. when the NAP conjugates are to be arrayed, the biochip substrates comprise an array of capture probes. By “capture probes” herein is meant nucleic acids (attached either directly or indirectly to the substrate as is more fully outlined below ) that are used to bind, e.g. hybridize, the NAP conjugates of the invention. Capture probes comprise nucleic acids as defined herein.

Capture probes are designed to be substantially complementary to capture sequences of the vectors, as is described below, such that hybridization of the capture sequence and the capture probes of the present invention occurs. As outlined below, this complementarity need not be perfect; there may be any number of base pair mismatches which will interfere with hybridization between the capture sequences and the capture probes of the present invention. However, if the number of mutations is so great that no hybridization can occur under even the least stringent of hybridization conditions, the sequence is not a complementary sequence. Thus, by “substantially complementary” herein is meant that the probes are sufficiently complementary to the capture sequences to hybridize under normal reaction conditions.

Nucleic acid arrays are known in the art, and include, but are not limited to, those made using photolithography techniques (Affymetrix GeneChip™), spotting techniques (Synteni and others), 25 printing techniques (Hewlett Packard and Rosetta), three dimensional “gel pad” arrays (U.S. Pat. No. 5,552,270), nucleic acid arrays on electrodes and other metal surfaces (WO 98/20162; WO 98/12430; WO 99/57317; and WO 01/07665) microsphere arrays (U.S. Pat. No. 6,023,540; WO 00/16101; WO 99/67641; and WO 00/39587), arrays made using functionalized materials (see PhotoLink™ technology from SurModics); all of which are expressly incorporated by reference.

As will be appreciated by those in the art, the capture probes or candidate ligands can be attached either directly to the substrate, or indirectly, through the use of polymers or through the use of microspheres.

Preferred methods of binding to the supports include the use of antibodies (which do not sterically block either the ligand binding site or activation sequence when the protein is bound to the support), direct binding to “sticky” or ionic supports, chemical crosslinking, the use of labeled components (e.g. the assay component is biotinylated and the surface comprises strepavidin, etc.) the synthesis of the target on the surface, etc. Following binding of the NAP conjugate or target molecule, excess unbound material is removed by suitable methods including, for example, chemical, physical, and biological separation techniques. The sample receiving areas may then be blocked through incubation with bovine serum albumin (BSA), casein or other innocuous protein or other moiety.

In a preferred embodiment, the ligands are attached to silica surfaces such as glass slides or glass beads, using techniques sometimes referred to as “small molecule printing” (SMP) as outlined in MacBeath et al., J. Am. Chem. Soc. 121(34):7967 (1999); Macbeath et al., Science 289:1760; Hergenrother et al., J. Am. Chem. Soc. 122(32):7849 (2000), all of which are expressly incorporated herein by reference. This generally relies on a maleimide derivatized glass slides. Thiol-containing compounds readily attach to the surface upon printing. In addition, a particular benefit of this system is the scarcity of non-specific protein binding to the surface, presumably due to the hydrophilicity of the maleimide functionality.

A preferred method of this embodiment uses traditional “split and mix” combinatorial synthesis of small molecule ligands, using beads for example. In many instances, as is known in the art, the beads can be “tagged” or “encoded” during synthesis. The attachment of the ligands to the beads is labile in some way, frequently either chemically cleavable or photocleavable. By releasing individual ligands into for example microtiter plates, these microtiter plates can be utilized in spotting techniques using standard spotters such as are used in nucleic acid microarrays as outlined herein.

**ID34

In addition, it should be noted that other types of support bound panning systems can be done. For example, either the candidate targets or the NAP conjugates can be attached to beads and screened against the other component. In one embodiment, the beads can be encoded or tagged using traditional methods, such as the incorporation of dyes or other labels, or nucleic acid “tags”. Alternatively, the beads can be encoded on the basis of physical parameters, such as bead size or composition, or combinations. For example, target analytes are attached to glass surfaces or beads, wherein a single glass bead size corresponds to a homogeneous population of molecules. Pools of different sized beads containing different targets are pooled, and the binding assays using the NAP conjugates are run. The beads are then sorted on the basis of size using any number of sizing techniques (meshing, filtering, etc.), and beads containing NAP conjugates can then identified, the NAP conjugates eluted, amplified, validated, etc.

As will be appreciated by those in the art, it is also possible to multiplex this system, multiple targets could be attached to the same size beads, and “hits” could then be deconvoluted later. Similarly, and in addition if desired, different coding schemes for beads can be used. For example, beads with magnetic cores in different sizes can be used, or dyes could be incorporated, etc.

In a preferred embodiment, the target molecule is bound to the support, and a NAP conjugate is added to the assay. Alternatively, the NAP conjugate is bound to the support and the target molecule is added. Novel binding agents include specific antibodies, non-natural binding agents identified in screens of chemical libraries, peptide analogs, etc. Of particular interest are screening assays for agents that have a low toxicity for human cells. Determination of the binding of the target and the candidate protein is done using a wide variety of assays, including, but not limited to labeled in vitro protein-protein binding assays, electrophoretic mobility shift assays, immunoassays for protein binding, the detection of labels, functional assays (phosphorylation assays, etc.) and the like.

The determination of the binding of the candidate protein to the target molecule may be done in a number of ways. In a preferred embodiment, one of the components, preferably the soluble one, is labeled, and binding determined directly by detection of the label. For example, this may be done by attaching the NAP conjugate to a solid support, adding a labeled target molecule (for example a target molecule comprising a fluorescent label), removing excess reagent, and determining whether the label is present on the solid support. This system may also be run in reverse, with the target (or a library of targets) being bound to the support and a NAP conjugate, preferably comprising a primary or secondary label, is added. For example, NAP conjugates comprising fusions with GFP or a variant may be particularly useful. Various blocking and washing steps may be utilized as is known in the art.

As will be appreciated by those in the art, it is also possible to contact the NAP conjugates and the targets prior to immobilization on a support.

In a preferred embodiment, the solid support is in an array format; that is, a biochip is used which comprises one or more libraries of either candidate agents, targets (including ligands such as small molecules) or NAP conjugates attached to the array. This can find particular use in assays for nucleic acid binding proteins, as nucleic acid biochips are well known in the art. In this embodiment, the nucleic acid targets are on the array and the NAP conjugates are added. Similarly, protein biochips of libraries of target proteins can be used, with labeled NAP conjugates added. Alternatively, the NAP conjugates can be attached to the chip, either through the nucleic acid or through the protein components of the system.

This may also be done using bead based systems; for example, for the detection of nucleic acid binding proteins, standard “split and mix” techniques, or any standard oligonucleotide synthesis schemes, can be run using beads or other solid supports, such that libraries of either sequences or candidate agents are made. The addition of NAP conjugate libraries then allows for the detection of candidate proteins that bind to specific sequences.

In some embodiments, only one of the components is labeled; alternatively, more than one component may be labeled with different labels.

In a preferred embodiment, the binding of the candidate protein is determined through the use of competitive binding assays. In this embodiment, the competitor is a binding moiety known to bind to the target molecule such as an antibody, peptide, binding partner, ligand, etc. Under certain circumstances, there may be competitive binding as between the target and the binding moiety, with the binding moiety displacing the target.

Thus, a preferred utility of the invention is to determine the components to which a drug will bind. That is, there are many drugs for which the targets upon which they act are unknown, or only partially known.

By starting with a drug, and NAP conjugates comprising a library of cDNA expression products from the cell type on which the drug acts, the elucidation of the proteins to which the drug binds may be elucidated. By identifying other proteins or targets in a signaling pathway, these newly identified proteins can be used in additional drug screens, as a tool for counterscreens, or to profile chemically induced events. Furthermore, it is possible to run toxicity studies using this same method; by identifying proteins to which certain drugs undesirably bind, this information can be used to design drug derivatives without these undesirable side effects. Additionally, drug candidates can be run in these types of screens to look for any or all types of interactions, including undesirable binding reactions. Similarly, it is possible to run libraries of drug derivatives as the targets, to provide a two-dimensional analysis as well.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound. Similarly, ELISA techniques are generally preferred.

A variety of other reagents may be included in the screening assays. These include reagents such as, but not limited to, salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, co-factors such as cAMP, ATP, etc., may be used. The mixture of components may be added in any order that provides for the requisite binding.

Screening for agents that modulate the activity of the target molecule may also be done. As will be appreciated by those in the art, the actual screen will depend on the identity of the target molecule. In a preferred embodiment, methods for screening for a candidate protein capable of modulating the activity of the target molecule comprise the steps of adding a NAP conjugate to a sample of the target, as above, and determining an alteration in the biological activity of the target. “Modulation” or “alteration” in this context includes an increase in activity, a decrease in activity, or a change in the type or kind of activity present. Thus, in this embodiment, the candidate protein should both bind to the target (although this may not be necessary), and alter its biological or biochemical activity as defined herein. The methods include both in vitro screening methods, as are generally outlined above, and ex vivo screening of cells for alterations in the presence, distribution, activity or amount of the target. Alternatively, a candidate peptide can be identified that does not interfere with target activity, which can be useful in determining drug-drug interactions.

Thus, in this embodiment, the methods comprise combining a target molecule and preferably a library of NAP conjugates and evaluating the effect on the target molecule's bioactivity. This can be done in a wide variety of ways, as will be appreciated by those in the art.

In these in vitro systems, e.g., cell-free systems, in either embodiment, e.g., in vitro binding or activity assays, once a “hit” is found, the NAP conjugate is retrieved to allow identification of the candidate protein. Retrieval of the NAP conjugate can be done in a wide variety of ways, as will be appreciated by those in the art and will also depend on the type and configuration of the system being used.

In a preferred embodiment, as outlined herein, a rescue tag or “retrieval property” is used. As outlined above, a “retrieval property” is a property that enables isolation of the fusion enzyme when bound to the target. For example, the target can be constructed such that it is associated with biotin, which enables isolation of the target-bound fusion enzyme complexes using an affinity column coated with streptavidin. Alternatively, the target can be attached to magnetic beads, which can be collected and separated from non-binding candidate proteins by altering the surrounding magnetic field. Alternatively, when the target does not comprise a rescue tag, the NAP conjugate may comprise the rescue tag. For example, affinity tags may be incorporated into the fusion proteins themselves. Similarly, the fusion enzyme-nucleic acid molecule complex can be also recovered by immunoprecipitation. Alternatively, rescue tags may comprise unique vector sequences that can be used to PCR amplify the nucleic acid encoding the candidate protein. In the latter embodiment, it may not be necessary to break the covalent attachment of the nucleic acid and the protein, if PCR sequences outside of this region (that do not span this region) are used.

In a preferred embodiment, after isolation of the NAP conjugate of interest, the covalent linkage between the fusion enzyme and its coding nucleic acid molecule can be severed using, for instance, nuclease-free proteases, the addition of non-specific nucleic acid, or any other conditions that preferentially digest proteins and not nucleic acids.

The nucleic acid molecules are purified using any suitable methods, such as those methods known in the art, and are then available for further amplification, sequencing or evolution of the nucleic acid sequence encoding the desired candidate protein. Suitable amplification techniques include all forms of PCR, OLA, SDA, NASBA, TMA, Q-βR, etc. Subsequent use of the information of the “hit” is discussed below.

In a preferred embodiment, the NAP conjugates are used in ex vivo screening techniques. In this embodiment, the expression vectors of the invention are introduced into host cells to screen for candidate proteins with a desired property, e.g., capable of altering the phenotype of a cell. An advantage of the present inventive method is that screening of the fusion enzyme library can be accomplished intracellularly. One of ordinary skill in the art will appreciate the advantages of screening candidate proteins within their natural environment, as opposed to lysing the cell to screen in vitro. In ex vivo or in vivo screening methods, variant peptides are displayed in their native conformation and are screened in the presence of other possibly interfering or enhancing cellular agents. Accordingly, screening intracellularly provides a more accurate picture of the actual activity of the candidate protein and, therefore, is more predictive of the activity of the peptide ex vivo or in vivo. Moreover, the effect of the candidate protein on cellular physiology can be observed. Thus, the invention finds particular use in the screening of eucaryotic cells.

Ex vivo and/or in vivo screening can be done in several ways. In a preferred embodiment, the target need not be known; rather, cells containing the expression vectors of the invention are screened for changes in phenotype. Cells exhibiting an altered phenotype are isolated, and the target to which the NAP conjugate bound is identified as outlined below, although as will be appreciated by those in the art and outlined herein, it is also possible to bind the fusion polypeptide and the target prior to forming the NAP conjugate. Alternatively, the target may be added exogeneously to the cell and screening for binding and/or modulation of target activity is done. In the latter embodiment, the target should be able to penetrate the membrane, by, for instance, direct penetration or via membrane transporting proteins, or by fusions with transport moieties such as lipid moieties or HIV-tat, described below.

In general, experimental conditions allow for the formation of NAP conjugates within the cells prior to screening, although this is not required. That is, the attachment of the NAM fusion enzyme to the EAS may occur at any time during the screening, either before, during or after, as long as the conditions are such that the attachment occurs prior to mixing of cells or cell lysates containing different fusion nucleic acids.

As will be appreciated by those in the art, the type of cells used in this embodiment can vary widely. Basically, any eucaryotic or procaryotic cells can be used, with mammalian cells being preferred, especially mouse, rat, primate and human cells. The host cells can be singular cells, or can be present in a population of cells, such as in a cell culture, tissue, organ, organ system, or organism (e.g., an insect, plant or animal). As is more fully described below, a screen will be set up such that the cells exhibit a selectable phenotype in the presence of a candidate protein. As is more fully described below, cell types implicated in a wide variety of disease conditions are particularly useful, so long as a suitable screen may be designed to allow the selection of cells that exhibit an altered phenotype as a consequence of the presence of a candidate agent within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be genetically engineered, that is, contain exogeneous nucleic acid, for example, to contain target molecules.

In a preferred embodiment, a first plurality of cells is screened. That is, the cells into which the expression vectors are introduced are screened for an altered phenotype. Thus, in this embodiment, the effect of the candidate protein is seen in the same cells in which it is made; i.e. an autocrine effect.

By a “plurality of cells” herein is meant roughly from about 10³ cells to 10⁸ or 10⁹, with from 10⁶ to 10⁸ being preferred. This plurality of cells comprises a cellular library, wherein generally each cell within the library contains a member of the NAP conjugate molecular library, i.e. a different candidate protein, although as will be appreciated by those in the art, some cells within the library may not contain an expression vector and some may contain more than one.

In a preferred embodiment, the expression vectors are introduced into a first plurality of cells, and the effect of the candidate proteins is screened in a second or third plurality of cells, different from the first plurality of cells, i.e. generally a different cell type. That is, the effect of the candidate protein is due to an extracellular effect on a second cell; i.e. an endocrine or paracrine effect. This is done using standard techniques. The first plurality of cells may be grown in or on one media, and the media is allowed to touch a second plurality of cells, and the effect measured. Alternatively, there may be direct contact between the cells. Thus, “contacting” is functional contact, and includes both direct and indirect. In this embodiment, the first plurality of cells may or may not be screened.

If necessary, the cells are treated to conditions suitable for the expression of the fusion nucleic acids (for example, when inducible promoters are used), to produce the candidate proteins.

Thus, the methods of the present invention preferably comprise introducing a molecular library of fusion nucleic acids or expression vectors into a plurality of cells, thereby creating a cellular library. Preferably, two or more of the nucleic acids comprises a different nucleotide sequence encoding a different candidate protein. The plurality of cells is then screened, as is more fully outlined below, for a cell exhibiting an altered phenotype. The altered phenotype is due to the presence of a candidate protein.

By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the candidate protein can change the phenotype of the cell in some detectable and/or measurable way.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc.

The present methods have utility in, for example, cancer applications. The ability to rapidly and specifically kill tumor cells is a cornerstone of cancer chemotherapy. In general, using the methods of the present invention, random or directed libraries (including cDNA libraries) can be introduced into any tumor cell (primary or cultured), and peptides identified which by themselves induce apoptosis, cell death, loss of cell division or decreased cell growth. This may be done de novo, or by biased randomization toward known peptide agents, such as angiostatin, which inhibits blood vessel wall growth. Alternatively, the methods of the present invention can be combined with other cancer therapeutics (e.g. drugs or radiation) to sensitize the cells and thus induce rapid and specific apoptosis, cell death, loss of cell division or decreased cell growth after exposure to a secondary agent. Similarly, the present methods may be used in conjunction with known cancer therapeutics to screen for agonists to make the therapeutic more effective or less toxic. This is particularly preferred when the chemotherapeutic is very expensive to produce such as taxol.

In a preferred embodiment, the present invention finds use with assays involving infectious organisms. Intracellular organisms such as mycobacteria, listeria, salmonella, pneumocystis, yersinia, leishmania, T. cruzi, can persist and replicate within cells, and become active in immunosuppressed patients. There are currently drugs on the market and in development which are either only partially effective or ineffective against these organisms. Candidate libraries can be inserted into specific cells infected with these organisms (pre- or post-infection), and candidate proteins selected which promote the intracellular destruction of these organisms in a manner analogous to intracellular “antibiotic peptides” similar to magainins. In addition peptides can be selected which enhance the cidal properties of drugs already under investigation which have insufficient potency by themselves, but when combined with a specific peptide from a candidate library, are dramatically more potent through a synergistic mechanism. Finally, candidate proteins can be isolated which alter the metabolism of these intracellular organisms, in such a way as to terminate their intracellular life cycle by inhibiting a key organismal event.

In a preferred embodiment, the compositions and methods of the invention are used to detect protein-protein interactions, similar to the use of a two-hybrid screen. This can be done in a variety of ways and in a variety of formats. As will be appreciated by those in the art, this embodiment and others outlined herein can be run as a “one dimensional” analysis or “multidimensional” analysis. That is, one NAP conjugate library can be run against a single target or against a library of targets. Alternatively, more than one NAP conjugate library can be run against each other.

In a preferred embodiment, the compositions and methods of the invention are used in protein drug discovery, particularly for protein drugs that interact with targets on cell surfaces.

In a preferred embodiment, as outlined above, the compositions and methods of the invention are used to discover DNA or nucleic acid binding proteins, using nucleic acids as the targets.

In a preferred embodiment, the libraries are pre-separated into sublibraries that are employed to identify specific enzymatic components within each sublibrary. In this embodiment, target analytes or ligands that are substrates, e.g. are modified by enzymes to release or generate a specific signal which may be detected, preferably optically (e.g. spectophotometrically, fluorescently, etc.). For example, phosphatases may be visualized by employing organophosphates, which when hydrolyzed release p-nitrophenol, which is monitored at 350 nm.

Thus, in this embodiment, the sublibraries are generated by diluting standard sized libraries (e.g. 10⁶) and then splitting the library into sublibrary pools. Each individual pool can then be independently transformed into host cells such as bacteria, amplifed and isolated. Each pool is then transfected individually into the host cells (preferably mammalian) of interest, lysed and the lysate placed into individual wells. The ligand substrates are then added, and “hits” identified optically and collected. This process may optionally be reiterated, followed by transformation of the well contents into bacterial cells and plated. Individual colonies are picked, the plasmids in vitro translated and the products treated with the ligand substrates. All active clones are then identified and characterized as outlined herein.

In a preferred embodiment, the compositions and methods of the invention are used to screen for NAM enzymes with decreased toxicity for the host cells. For example, Rep proteins of the invention can be toxic to some host cells. The present inventive methods can be used to identify or generate Rep proteins with decreased toxicity. In this particular embodiment, Rep variants or, in an alternative, random peptides are used in the present inventive conjugates to observe cell toxicity and binding affinity to an EAS.

With respect to EASs, the present inventive methods can also be utilized to identify novel or improved EASs for use in the present inventive expression vectors. An EAS for a particular NAM enzyme of interest can also be identified using the present inventive method. Formation of covalent structure of NAM enzyme and EAS can determined using suitable methods that are present in the art, e.g. those described in U.S. Pat. No. 5545529. In general, the candidate NAM enzyme can be expressed using a variety of hosts, such as bacteria or mammalian cells. The expressed protein can then be tested with candidate DNA sequences, such a library of fragments obtained from the genome from which the NAM enzyme is cloned. Contacts between the NAM enzyme and with the library of DNA fragments under appropriate conditions (such as inclusion of cofactors) allow for the formation of covalent NAM enzyme-DNA conjugates. The mixture can then be separated using a variety of techniques. The isolated bound nucleic acid sequences can then be identified and sequenced. These sequences can be tested further via a variety of mutagenesis techniques. The confirmed sequence motif can then be used an EAS.

In a preferred embodiment, the compositions and methods of the invention are used in pharmacogenetic studies. For example, by building libraries from individuals with different phenotypes and testing them against targets, differential binding profiles can be generated. Thus, a preferred embodiment utilizes differential binding profiles of NAP conjugates to targets to elucidate disease genes, SNPs or proteins.

The present invention also finds use in screening for bioactive agents on the surface of cells, viruses and microbial organisms, as well as on the surface of subcellular organelles. these bioactive targets, which may be native to the organism or displayed via recombinant molecular techniques, can be aimed for gene therapy or antibody therapy, especially if they are disease related or disease specific. For example, there are a wide variety of cell surface receptors known to be involved in disease states such as cancer.

In this embodiment, the NAP conjugate library is made, preferably using a candidate protein library derived from a cDNA library from an interesting tissue, such as peripheral blood cells, bone marrow, spleen and thymus from patients carrying or exhibiting the disease. For example, it may be of use to evaluate immunoglobulins, cytokines, T or B cell receptors, surface proteins of natural killer cells, etc. Of course, additional tissues as outlined herein can also be used, particularly from tissues involved in the disease state.

The cell lysates of the cells are formed as outlined herein, or in vitro translation systems can be used, and the library of NAP conjugates purified if necessary. This can be done as outlined herein, using for example an anti-NAM enzyme antibodies, purification or rescue tags and epitopes, etc.

The NAP conjugate library can then optionally be pre-screened or filtered by passing it thorugh cells or other particles suitable for absorbing non-specific binding partners, which express the common or housekeeping proteins of the disease cells but lack the disease specific targets. After “cleaning”, the NAP conjugate library is incubated with the disease cells. After optional washing, the bound fraction of the NAP conjugate library can be eluted, amplified, identified and/or characterized as outlined herein. The eluted material is used for sequence analysis or for a reiterative round of panning.

Alternatively, in the case where a lower amount of disease-specific target is also expressed on the surface of normal cells, the screening procedure can be reversed for a few rounds. That is, the NAP conjugate library is first incubated with the disease cells and the non-specific binders are competed off with normal cells. The specific binders of the library are then eluted from the disease cells.

In addition, the NAP conjugate library can also be used for screening proteins causing phenotypic changes such as overproduction or inhibition of protein expression. The bound candidate proteins are eluted from the altered phenotype cells after separation from the parent cells by specific antibodies or cell sorting. The phenotypic screening is applied to disease cells to discover candidate proteins that alter the growth of disease cells. Similarly, this type of screening can be applied to normal cells to identify proteins that switch cells to certain pathways, such as a disease pathway. Furthermore, other organisms or tissues can also be used to search for candidate proteins that can bind and/or alter the growth of the targets, including viruses, cells, microbial organisms, cell lines, tissue or tissue sections such as endothelial cell monolayers, cardiac muscle sections, or solid tumor sections. When virsues are used as the target analytes, the NAP conjugate library screening is used to identify proteins that alter attachment, infectivity, etc. of the virus. Similarly, instead of viruses as the target, subcellular organelles such as the nucleus, ribosomes, mitrochondria, chloroplasts, endoplasmic reticulum and Golgi apparatus from any number of different cells, as outlined herein, can be used.

As will be appreciated by those in the art, there are a wide variety of possible primary and secondary screens which may be performed using the present invention. For example, many of the screens and panning techniques outlined herein utilize a single entity (e.g. target analyte) for screening against the NAP conjugate libraries or cells comprising those libraries. However, sometimes the observed biological effect exerted by a compound of interest is dependent upon that compound's ability to effect or affect oligomerization of particular proteins. These types of interactions may not be readily identified in a primary screen, as many of the methods rely upon the covalent conjugation of the compound of interest to a tag in which the tag can be used to isolate, using affinity binding, the binding partners. If the linker or tag interferes with the subsequent protein binding to the compound-protein complex, that information may not be observed. Accordingly, in a preferred embodiment, a secondary screening protocol may be run.

In general, this process is outlined as follows. The first primary screen is run, using a tagged compound of interest panned against a library of NAP conjugates. This tagged compound is used to isolate all candidate proteins that bind to it. By decoding the cDNA of the isolated candidates, all possible candidates for the secondary screen are identified. The secondary screen then is initiated by directly or indirectly covalently linking the primary candidate hits to a solid support, using any number of known techniques such as those outlined herein. In general, the linkage technique should not interfere with the binding site of the original tagged compound, and should maximize the ability of the protein to interact with other proteins. In some instances, a variety of different linkages and/or linkage sites are used, and may include the additional use of linkers as outlined herein.

The secondary screen proceeds with the incubation of the array of attached candidate proteins with the original compound of interest, preferably in an untagged form, in the presence of a NAP conjugate library. In a preferred embodiment, to minimize the background signals, the NAP conjugate library may be first incubated with the candidate protein linked to a solid support (in the absence of the ligand), and all entities that are not retained on the solid support are used in the screen. Subsequent isolation and decoding of the cDNA of the candidate proteins that bind the protein-ligand complex thus identifies additional interactions mediated by the ligand.

In a preferred embodiment, once a cell with an altered phenotype is detected, the cell is isolated from the plurality which do not have altered phenotypes. This may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

In a preferred embodiment, as outlined above, the NAP conjugate is isolated from the positive cell. This may be done in a number of ways. In a preferred embodiment, primers complementary to DNA regions common to the NAP constructs, or to specific components of the library such as a rescue sequence, defined above, are used to “rescue” the unique candidate protein sequence. Alternatively, the candidate protein is isolated using a rescue sequence. Thus, for example, rescue sequences comprising epitope tags or purification sequences may be used to pull out the candidate protein, using immunoprecipitation or affinity columns. In some instances, as is outlined below, this may also pull out the primary target molecule, if there is a sufficiently strong binding interaction between the candidate protein and the target molecule. Alternatively, the peptide may be detected using mass spectroscopy. Once rescued, the sequence of the candidate protein and fusion nucleic acid can be determined. This information can then be used in a number of ways, i.e., genomic databases.

For in vitro, ex vivo, and in vivo screening methods, once the “hit” has been identified, the results are preferably verified. As will be appreciated by those in the art, there are a variety of suitable methods that can be used. In a preferred embodiment, the candidate protein is resynthesized and reintroduced into the target cells, to verify the effect. This may be done using recombinant methods, e.g. by transforming naive cells with the expression vector (or modified versions, e.g. with the candidate protein no longer part of a fusion), or alternatively using fusions to the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In addition, for both in vitro and ex vivo screening methods, the process may be used reiteratively. That is, the sequence of a candidate protein is used to generate more candidate proteins. For example, the sequence of the protein may be the basis of a second round of (biased) randomization, to develop agents with increased or altered activities. Alternatively, the second round of randomization may change the affinity of the agent. Furthermore, if the candidate protein is a random peptide, it may be desirable to put the identified random region of the agent into other presentation structures, or to alter the sequence of the constant region of the presentation structure, to alter the conformation/shape of the candidate protein.

The methods of using the present inventive library can involve many rounds of screenings in order to identify a nucleic acid of interest. For example, once a nucleic acid molecule is identified, the method can be repeated using a different target. Multiple libraries can be screened in parallel or sequentially and/or in combination to ensure accurate results. In addition, the method can be repeated to map pathways or metabolic processes by including an identified candidate protein as a target in subsequent rounds of screening.

In a preferred embodiment, the candidate protein is used to identify target molecules, i.e. the molecules with which the candidate protein interacts. As will be appreciated by those in the art, there may be primary target molecules, to which the protein binds or acts upon directly, and there may be secondary target molecules, which are part of the signaling pathway affected by the protein agent; these might be termed “validated targets”.

In a preferred embodiment, the candidate protein is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Or, peptides can be used as “bait” in either yeast or mammalian two or three hybrid systems. Such interaction cloning approaches have been very useful to isolate DNA-binding proteins and other interacting protein components. The peptide(s) can be combined with other pharmacologic activators to study the epistatic relationships of signal transduction pathways in question. It is also possible to synthetically prepare labeled peptides and use it to screen a cDNA library expressed in bacteriophage for those cDNAs which bind the peptide.

Once primary target molecules have been identified, secondary target molecules may be identified in the same manner, using the primary target as the “bait”. In this manner, signaling pathways may be elucidated. Similarly, protein agents specific for secondary target molecules may also be discovered, to allow a number of protein agents to act on a single pathway, for example for combination therapies.

In a preferred embodiment, the methods and compositions of the invention can be performed using a robotic system. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

A wide variety of automatic components can be used to perform the present inventive method or produce the present inventive compositions, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4 C to 100° C.

In a preferred embodiment, interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some preferred embodiments, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems, for cell culture growth and transformation in multi-well plates or tubes and for hazardous operations. The living cells will be grown under controlled growth conditions, with controls for temperature, humidity, and gas for time series of the live cell assays. Automated transformation of cells and automated colony pickers will facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which can be done using any number of known heating and cooling systems, including Peltier systems.

In a preferred embodiment, the robotic apparatus includes a central processing unit which communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

The above-described methods of screening a pool of fusion enzyme-nucleic acid molecule complexes for a nucleic acid encoding a desired candidate protein are merely based on the desired target property of the candidate protein. The sequence or structure of the candidate proteins does not need to be known. A significant advantage of the present invention is that no prior information about the candidate protein is needed during the screening, so long as the product of the identified coding nucleic acid sequence has biological activity, such as specific association with a targeted chemical or structural moiety. The identified nucleic acid molecule then can be used for understanding cellular processes as a result of the candidate protein's interaction with the target and, possibly, any subsequent therapeutic or toxic activity.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

All references cited herein are incorporated by reference.

What is claimed is:

1. A library of nucleic acid/protein (NAP) conjugates each comprising:
a) a fusion polypeptide comprising:
i) a NAM enzyme; and
ii) a candidate protein;
b) an expression vector comprising
i) a fusion nucleic acid comprising:
1) a nucleic acid encoding said NAM enzyme; and
2) a nucleic acid encoding said candidate protein;
wherein at least two of said candidate proteins are different; and,
c) an enzyme attachment sequence (EAS), wherein said EAS is an RNA sequence; wherein said EAS and said NAM enzyme are covalently attached.

2. A library of expression vectors each comprising:
a) a fusion nucleic acid comprising:
i) a nucleic acid encoding a NAM enzyme; and,
ii) a nucleic acid encoding a candidate protein;
wherein at least two of said candidate proteins are different; and,
b) a DNA binding motif that is recognized by a small molecule conjugate.

3. A library according to claim 1 or 2 wherein said NAM enzyme is a Rep protein.

4. A library according to claim 1 or 2 wherein said Rep protein is a Rep 68 protein.

5. A library according to claim 1 or 2 wherein said Rep protein is a Rep 78 protein.

6. A method of making a library of fusion polypeptides comprising:
a) providing a first fusion nucleic acid comprising:
i) a nucleic acid encoding a NAM enzyme; and
ii) a nucleic acid encoding a ligation mediating moiety;
b) providing a second fusion nucleic acid comprising:
i) a nucleic acid encoding a candidate protein; and
ii) a nucleic acid encoding a ligation substrate;
wherein at least two of said candidate proteins are different;
c) ligating said first and said second fusion nucleic acids to form fusion nucleic acids comprising a Rep protein and a candidate protein; and,
d) expressing said fusion nucleic acids under conditions whereby a library of fusion polypeptides are formed wherein said fusion polypeptides comprise a NAM enzyme and a candidate protein.

7. A method according to claim 6 wherein said ligation substrate is ubiquitin.

8. A method of making a library of fusion polypeptides comprising:
a) providing a first fusion nucleic acid comprising:
i) a nucleic acid encoding a NAM enzyme; and
ii) a nucleic acid encoding an N-terminal intein motif;
b) providing a second fusion nucleic acid comprising:
i) a nucleic acid encoding a candidate protein; and
ii) a nucleic acid encoding a C-terminal intein motif;
wherein at least two of said candidate proteins are different.
c) combining said first and said second fusion nucleic acids under conditions whereby protein splicing occurs; and,
d) forming a library of fusion polypeptides comprising a NAM enzyme and a candidate protein.

9. A method of making a library of fusion polypeptides comprising:
a) providing:
i) an acceptor donor substrate comprising a NAM enzyme wherein said NAM enzyme comprises at least one reactive glutamine residue;
ii) a donor candidate protein comprising at least one lysine residue;
b) combining said NAM enzyme and said candidate protein under conditions whereby transglutaminase is active; and,
c) forming a NAM enzyme-candidate protein fusion.

10. A library of expression vectors comprising:
a) a fusion nucleic acid comprising:
i) a nucleic acid encoding a NAM enzyme; and
ii) a nucleic acid encoding a candidate protein;
b) an enzyme attachment sequence (EAS) that is recognized by said NAM enzyme; and
c) a recombination system.

11. A method of detecting the presence of a target analyte in a sample comprising:
a) providing a biochip comprising an array of candidate target analytes;
b) contacting said array with a library of nucleic acid/protein (NAP) conjugates comprising:
i) a fusion polypeptide comprising:
1) a NAM enzyme; and
2) a candidate protein;
ii) an expression vector comprising:
1) a fusion nucleic acid comprising:
A) nucleic acid encoding said NAM enzyme;
B) nucleic acid encoding said candidate protein; and
C) an enzyme attachment sequence (EAS);
wherein said EAS and said NAM enzyme are covalently attached, under conditions wherein at least one of said candidate target analytes can bind to at least one of said candidate proteins to form an assay complex; and
c) detecting the presence of said assay complex on said substrate.
